(19) United States
(12) Patent Application Publication
(10) Pub. No.: US 2026/0258031 A1
(43) Pub. Date: Sep. 3, 2026

(54) CRYSTALLINE FORMS OF AN ESTROGEN RECEPTOR ANTAGONIST

(71) Applicant: Olema Pharmaceuticals, Inc., San Francisco, CA (US)

(72) Inventors: David C. Myles, Berkeley, CA (US); Rampurna Prasad Gullapalli, San Diego, CA (US); Jing Jim Zhang, San Francisco, CA (US); David Askin, Warren, NJ (US); Ekaterina Albert, West Lafayette, IN (US); Liping Wang, Tianjin (CN); Lina Yang, Tianjin (CN); Shuyan Huang, Tianjin (CN)

(21) Appl. No.: 18/867,232

(22) PCT Filed: May 19, 2023
(86) PCT No.: PCT/US23/22984

§ 371 Date: Nov. 19, 2024

(30) Foreign Application Priority Data
May 20, 2022 (WO) PCT/CN2022/094231

(51) Int. Cl. C07D 471/04

(52) U.S. Cl. CPC C07D 471/04

(57) ABSTRACT

The present disclosure provides crystalline solid forms of an estrogen receptor (ER) inhibitor, compositions thereof and methods of treating a ER-mediated disorder.

### RELATED APPLICATIONS

This application claims priority to and benefit of PCT Application No. PCT/CN2022/094231, filed May 20, 2022, the entire contents of which is hereby incorporated by reference.

### BACKGROUND

The estrogen receptor (ER) plays important roles in various cancers, including breast cancers. A variety of treatments have been developed to target the estrogen receptor and/or its activities.

### SUMMARY

There remains a need for anti-estrogen agents that can completely inhibit estrogen receptors, including those coded for by both wild-type and mutant versions (e.g., those containing activating mutations) of the gene encoding Estrogen Receptor-alpha (ERα), Estrogen Receptor 1 (ESR1). Selective estrogen receptor modulators (SERMs) or degraders (SERDs) are a particularly useful or promising tools for such therapy. Recently, classes of estrogen receptor antagonists, termed Complete Estrogen Receptor Antagonists (CERANs) have emerged as promising therapies for completely inhibiting the estrogen receptor.

CERANs are considered “complete” as compared to other estrogen receptor antagonists because they inactivate two distinct transcriptional activation functions (AF1 and AF2) of the estrogen receptor. Previous therapies that are not CERANs fail when activation mutations in the gene that codes for estrogen receptor 1 allows for activation of both AF1 and AF2 even in the absence of estrogen. The present disclosure provides salts, solid forms, and compositions and uses thereof of a compound useful for complete antagonism of the estrogen receptor, providing an option for treatment for subjects suffering from a cancer, and/or wherein the subject carries a mutation of estrogen receptor 1 (ESR1).

The compound (1R,3R)-2-(2-fluoro-2-methylpropyl)-3-methyl-1-(4-((1-propylazetidin-3-yl)oxy)phenyl)-2,3,4,9-tetrahydro-1H-pyrido[3,4-b]indole (“Compound 1”):

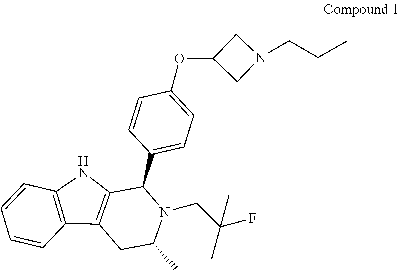 [SMILES: CCCN1CC(Oc2ccc([C@@H]3c4[nH]c5ccccc5c4C[C@@H](C)N3CC(C)(C)F)cc2)C1]

is a complete estrogen receptor antagonist published in PCT Publication No. WO 2017/059139 (the entire contents of which are hereby incorporated by reference), designated as Compound B. There remains a need for identifying salt, solid, hydrate and/or solvate forms of Compound 1 useful for various therapeutic applications.

During process development of Compound 1, Applicant recognized that, when preparing a solid dosage form (e.g., tablet or capsule) comprising Compound 1, the amorphous form of Compound 1 exhibited certain properties, e.g., flow properties, bulk density, and handleability, which made the process for generating a solid dosage form comprising Compound 1 difficult.

Moreover, certain attempts to formulate Compound 1 into a form more amenable for manufacturing and formulation, instead resulted in a form of Compound 1 that was solvated with certain, potentially toxic, organic solvents. Applicant recognized that Compound 1 in both free base and salt forms exhibits a high propensity toward solvation, which presents certain challenges when attempting to develop an unsolvated or hydrated form of Compound 1. As shown in Examples 6-9, polymorph screening experiments identified no unsolvated or hydrate forms of Compound 1 free base, and only a few unsolvated or hydrate salt forms of Compound 1.

The present disclosure, however, solves the problems identified above and provides a form that, in some embodiments, exhibits desirable characteristics such as improved stability, hygroscopicity, flow properties, compressibility, case of processing, consistency in manufacturing, particle size distribution, bulk density, pharmacokinetics, bioavailability, and ease of formulation. For example, the present disclosure encompasses the recognition that certain crystalline solid forms of Compound 1 as a fumarate salt (“Compound 1 Fumarate”) may be useful in compositions and methods described herein:

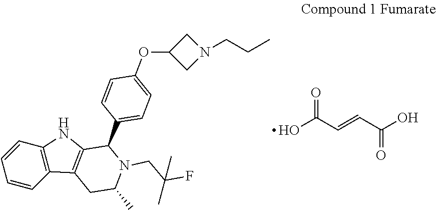 [SMILES: CCCN1CC(Oc2ccc([C@@H]3c4[nH]c5ccccc5c4C[C@@H](C)N3CC(C)(C)F)cc2)C1 ; O=C(O)/C=C/C(=O)O]

In some embodiments, the present disclosure provides unsolvated crystalline solid forms of Compound 1 Fumarate. In some embodiments, the present disclosure provides hydrated crystalline solid forms of Compound 1 Fumarate.

In some embodiments, the present disclosure provides a Compound 1 Fumarate Form E, as described herein.

In some embodiments, the present disclosure provides methods of inhibiting the estrogen receptor, or a mutation thereof, in a biological sample comprising contacting said biological sample with an estrogen receptor antagonist (e.g., Compound 1 Fumarate Form E).

In some embodiments, the present disclosure provides compositions comprising one or more forms of Compound 1 or Compound 1 Fumarate provided herein. In some embodiments, the present disclosure provides pharmaceutical compositions comprising one or more forms of Compound 1 or Compound 1 Fumarate provided herein and a pharmaceutically acceptable carrier.

In some embodiments, the present disclosure provides methods of treating patients or subjects suffering from a cancer related to the estrogen receptor or mutations of the estrogen receptor, comprising administering an estrogen receptor antagonist (e.g., Compound 1 Fumarate Form E).

In some embodiments, the present disclosure provides methods of treating estrogen receptor (ER)-associated diseases, disorders, and conditions (e.g., cancer) and/or for otherwise modulating (e.g., inhibiting) the estrogen receptor in the brain, comprising administering an estrogen receptor antagonist (e.g., Compound 1 Fumarate Form E).

In some embodiments, the present disclosure provides methods of treating an ER-associated disease disorder or condition (e.g., an ER-associated cancer, including but not limited to one that is or comprises tumor(s) in the brain such as brain metastases) by administering a particular complete estrogen receptor antagonist (e.g., Compound 1 Fumarate Form E) according to a regimen that achieves preferential accumulation in tumor relative to plasma in the patient (i.e., achieves accumulation in tumor to a concentration above that in plasma).

### BRIEF DESCRIPTION OF THE DRAWINGS

FIG. 1 is an XRPD pattern of Compound 1 Fumarate Form E.

FIG. 2 is a TGA curve (top) and a DSC curve (bottom) of Compound 1 Fumarate Form E.

FIG. 3 is an XRPD pattern of Compound 1 Fumarate Form E.

FIG. 4 is a TGA curve of Compound 1 Fumarate Form E.

FIG. 5 is a DSC curve of Compound 1 Fumarate Form E.

FIG. 6 is a DVS plot of Compound 1 Fumarate Form E.

FIG. 7 is a series of XRPD patterns from competitive slurry experiments of Compound 1 Fumarate Form E and Compound 1 Fumarate Form A Anhydrate in isopropanol.

FIG. 8 is a series of XRPD patterns from competitive slurry experiments of Compound 1 Fumarate Form E and Compound 1 Fumarate Form A Anhydrate in water.

FIG. 9 is a series of XRPD patterns from competitive slurry experiments of Compound 1 Fumarate Form E and Compound 1 Fumarate Form A Anhydrate in ethyl acetate.

### DETAILED DESCRIPTION

### Compound 1

Compound 1 is a complete estrogen receptor antagonist, published in PCT Publication No. WO 2017/059139 (the entirety of which is incorporated herein by reference), designated as Compound B. Exemplary methods for using Compound 1 are described in PCT Publication Nos. WO 2021/007146 and WO 2021/178846, the entirety of each of which are incorporated herein by reference.

A synthesis of Compound 1 is described in detail in Example 10 of WO 2017/059139, as well as in Example 1 herein.

### Compound 1 Fumarate

In some embodiments, the present disclosure provides a crystalline solid form of Compound 1 Fumarate, e.g., Compound 1 Fumarate Form E. Compound 1 Fumarate Form E is one of multiple polymorphic solid forms of Compound 1 Fumarate. As used herein, the term “polymorph” refers to the ability of a compound to exist in one or more different crystal structures. For example, one or more polymorphs may vary in pharmaceutically relevant physical properties between one form and another, e.g., solubility, stability, and/or hygroscopicity.

In some embodiments, Compound 1 Fumarate Form E exists in an unsolvated form. A crystalline solid form that does not have any water or solvent incorporated into the crystalline structure is “unsolvated.” In some embodiments, Compound 1 Fumarate Form E exists as an anhydrate. A crystalline solid form that does not have any water incorporated into the crystalline structure is an “anhydrate.” In some embodiments, Compound 1 Fumarate Form E is an unsolvated anhydrate.

In some embodiments, a crystalline form of Compound 1 Fumarate exists as a solvate and/or hydrate. As used herein, the term “solvate” refers to a solid form with a stoichiometric or non-stoichiometric amount of one or more solvents incorporated into the crystal structure. For example, a solvated or heterosolvated polymorph can comprise 0.05, 0.1, 0.2, 0.5, 1.0, 1.5, 2.0, etc. equivalents independently of one or more solvents incorporated into the crystal lattice. As used herein, the term “hydrate” refers to a solvate, wherein the solvent incorporated into the crystal structure is water.

It will be appreciated that “Compound 1 Fumarate” refers to a complex form comprising Compound 1 non-covalently associated with the co-former fumaric acid. Such non-covalent associations include, by way of example, ionic interactions, dipole-dipole interactions, π-stacking interactions, hydrogen bond interactions, etc. It will be appreciated that the term “Compound 1 Fumarate” encompasses salt forms resulting from an ionic interaction between Compound 1 and fumaric acid, as well as non-ionic associations between Compound 1 and fumaric acid.

As provided herein, Compound 1 Fumarate Form E has distinct XRPD peaks that are not reported in previous disclosures of Compound 1. As used herein, the term “about” when used in reference to a degree 2-theta value refers to the state value±0.2 degrees 2-theta.

In some embodiments, the present disclosure provides a complex form comprising Compound 1 and fumaric acid (i.e., Compound 1 Fumarate), wherein the complex form is Compound 1 Fumarate Form E. In some embodiments, Compound 1 Fumarate Form E comprises a 1:1 ratio of fumaric acid to Compound 1. In some embodiments, Compound 1 Fumarate Form E is an anhydrate.

In some embodiments, provided forms (e.g., forms of Compound 1 and Compound 1 Fumarate) are characterized by having peaks in its XRPD pattern selected from “substantially all” of a provided list, optionally within ±0.2 degrees 2-theta of the stated value. It will be appreciated that an XRPD pattern having “substantially all” of a provided list of peaks refers to an XRPD pattern that comprises at least 80% (e.g., 80%, 85%, 90%, 95%, 99% or 100%) of the listed peaks. In some embodiments, an XRPD pattern comprises at least 90% of the listed peaks. In some embodiments, an XRPD pattern comprises all of the listed peaks. In some embodiments, an XRPD pattern comprises all but one of the listed peaks. In some embodiments, an XRPD pattern comprises all but two of the listed peaks. In some embodiments, an XRPD pattern comprises all but three of the listed peaks.

In some embodiments, provided forms (e.g., forms of Compound 1 and Compound 1 Fumarate) are characterized by having a pattern or spectrum that is “substantially similar” to a Figure provided herein. It will be appreciated that a pattern or spectrum having “substantial similarity” to a Figure provided herein is one that comprises one or more features (e.g., position (degrees 2-theta) values, temperature values, % weight loss values, intensity, shape of curve, etc.) of the provided Figure so as to enable identification of the form (e.g., solid and/or salt form) characterized by the pattern or spectrum as being the same as the form characterized in the Figure. For example, in some embodiments, an XRPD pattern having substantial similarity to a provided Figure is one that comprises substantially all of the same peaks, optionally within ±0.2 degrees 2-theta of peaks in the reference Figure. In some embodiments, an XRPD pattern having substantial similarity to a provided Figure is one that comprises substantially all of the same peaks, optionally within ±0.2 degrees 2-theta of peaks in the reference Figure, with about the same intensities.

In some embodiments, Compound 1 Fumarate Form E is characterized by one or more peaks in its XRPD pattern selected from those at about 5.83, about 7.03, about 8.69, about 12.88, about 13.43, about 14.68, about 15.65, about 16.65, and about 18.46 degrees 2-theta. In some embodiments, Compound 1 Fumarate Form E is characterized by two or more peaks in its XRPD pattern selected from those at about 5.83, about 7.03, about 8.69, about 12.88, about 13.43, about 14.68, about 15.65, about 16.65, and about 18.46 degrees 2-theta. In some embodiments, Compound 1 Fumarate Form E is characterized by three or more peaks in its XRPD pattern selected from those at about 5.83, about 7.03, about 8.69, about 12.88, about 13.43, about 14.68, about 15.65, about 16.65, and about 18.46 degrees 2-theta.

In some embodiments, Compound 1 Fumarate Form E is characterized by peaks in its XRPD pattern at about 5.83, about 7.03, about 8.69, about 12.88, about 13.43, about 14.68, about 15.65, about 16.65, and about 18.46 degrees 2-theta. In some embodiments, Compound 1 Fumarate Form E is characterized by peaks in its XRPD pattern at substantially all of:

Position ± 0.2 (degrees 2-Theta)

5.83
7.03
8.69
10.91
12.88
13.43
14.68
15.65
16.08
16.65
17.72
18.46
18.88
19.63
19.99
21.73
22.00
22.31
23.83
24.63
25.02
26.48
27.37
28.60
29.32
29.81
30.05
35.69
39.70
40.32
40.97

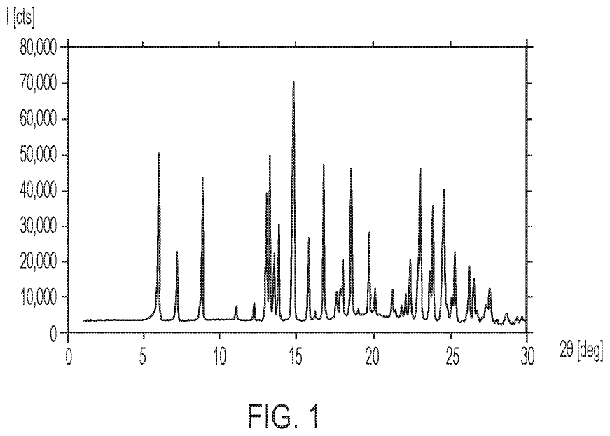

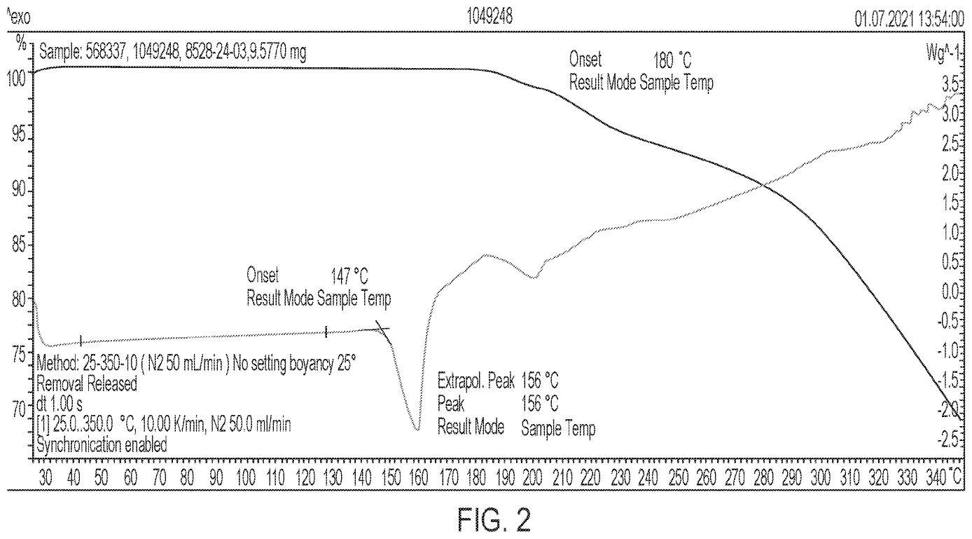

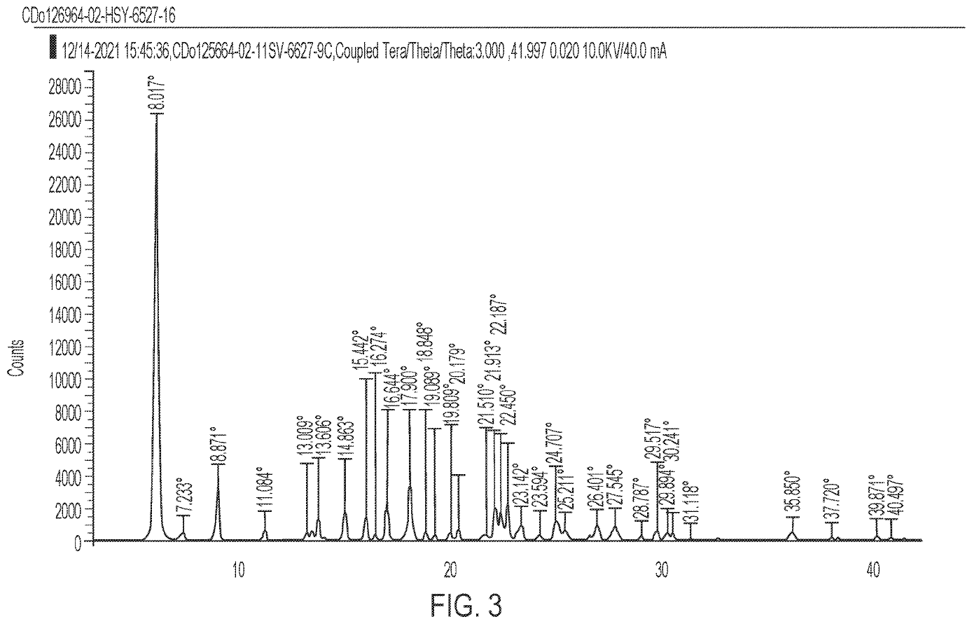

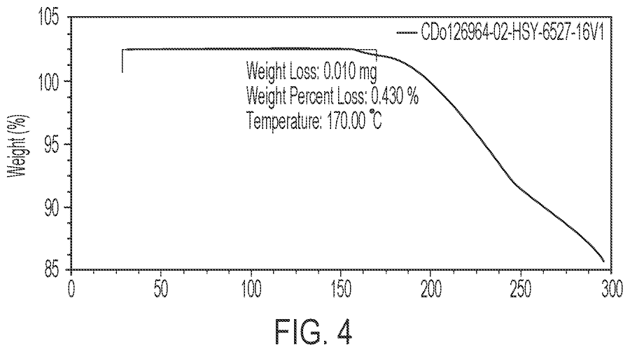

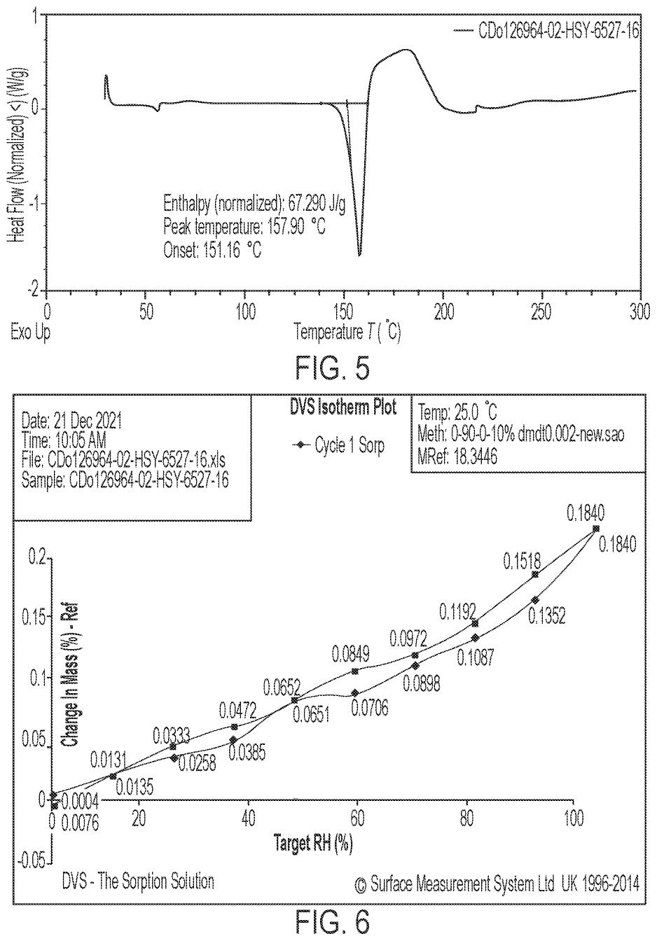

In some embodiments, Compound 1 Fumarate Form E is characterized by one or more of the following:
(i) an XRPD pattern substantially similar to that depicted in FIG. 1 and/or FIG. 3;
(ii) a TGA pattern substantially similar to that depicted in FIG. 2 and/or FIG. 4; and
(iii) a DSC pattern substantially similar to that depicted in FIG. 2 and/or FIG. 5.

### Preparing Provided Forms

In some embodiments, the present disclosure provides methods of preparing provided solid forms, e.g., Compound 1 Fumarate Form E. In some embodiments, Compound 1 Fumarate Form E is prepared by contacting Compound 1 (e.g., amorphous Compound 1, crystalline Compound 1, or a mixture thereof) with fumaric acid. In some embodiments, the present disclosure provides a method of preparing Compound 1 Fumarate Form E comprising steps of providing Compound 1; and combining Compound 1 with fumaric acid, optionally in a suitable solvent, to provide Compound 1 Fumarate Form E. In some embodiments, about 1.0, about 1.1, about 1.2, or about 2.0 equivalents of fumaric acid are added.

In some embodiments, Compound 1 Fumarate Form E is prepared by dissolving Compound 1 Fumarate (e.g., amorphous Compound 1 Fumarate, crystalline Compound 1 Fumarate, or a mixture thereof) in a suitable solvent and then causing Compound 1 Fumarate to return to the solid phase. In some embodiments, Compound 1 Fumarate Form E is prepared by combining Compound 1 Fumarate (e.g., amorphous Compound 1 Fumarate, crystalline Compound 1 Fumarate, or a mixture thereof) in a suitable solvent under suitable conditions and isolating Compound 1 Fumarate Form E.

In some embodiments, a suitable solvent is selected from 2-butanol, dichloroethane, ethanol, heptane, isopropanol, N-methylpyrrolidone, and water, or any combination thereof.

In some embodiments, a method of preparing Compound 1 Fumarate Form E comprises a step of heating a mixture comprising Compound 1 Fumarate to a suitable temperature (e.g., from about 30° C. to about 60° C.). In some embodiments, a method of preparing Compound 1 Fumarate Form E comprises a step of stirring a mixture comprising Compound 1 Fumarate at ambient temperature. In some embodiments, a method of preparing Compound 1 Fumarate Form E comprises a step of cooling a mixture comprising Compound 1 Fumarate to a suitable temperature (e.g., from about −20° C. to about 0° C.).

In some embodiments, Compound 1 Fumarate Form E precipitates from a mixture (e.g., a solution, suspension, or slurry). In some embodiments, Compound 1 Fumarate Form E crystallizes from a solution. In some embodiments, Compound 1 Fumarate Form E crystallizes from a solution following seeding of the solution (e.g., adding crystals of Compound 1 Fumarate Form E to the solution). In some embodiments, Compound 1 Fumarate Form E precipitates or crystallizes from a mixture after cooling, addition of an anti-solvent, and/or removal of all or part of a solvent through methods such as evaporation, distillation, filtration, reverse osmosis, absorption, or reaction.

In some embodiments, a method of preparing Compound 1 Fumarate Form E comprises a step of isolating Compound 1 Fumarate Form E. It will be appreciated that Compound 1 Fumarate Form E may be isolated by any suitable means. In some embodiments, Compound 1 Fumarate Form E is separated from a supernatant by filtration. In some embodiments, Compound 1 Fumarate Form E is separated from a supernatant by decanting.

In some embodiments, isolated Compound 1 Fumarate Form E is dried (e.g., in air or under reduced pressure, optionally at elevated temperature).

In some embodiments, Compound 1 Fumarate Form E is prepared by converting a solid form of Compound 1 Fumarate into Compound 1 Fumarate Form E.

In some embodiments, Compound 1 Fumarate Form E is prepared by a process comprising a step of combining Compound 1 (e.g., amorphous Compound 1) in a suitable solvent (e.g., isopropanol) with stirring at a suitable temperature (e.g., about 40° C.). In some embodiments, the process further comprises adding a first portion (e.g., about 0.5 equiv) of fumaric acid. In some embodiments, the process further comprises adding seed crystals of Compound 1 Fumarate Form E. In some embodiments, the process further comprises adding a second, third, and/or fourth portion (e.g., about 0.2-0.3 equiv) of fumaric acid. In some embodiments, the process further comprises adding a suitable anti-solvent (e.g., heptane). In some embodiments, the process further comprises cooling the mixture to ambient temperature (e.g., about 25° C.). In some embodiments, the process further comprises isolating a solid form of Compound 1 Fumarate Form E by a method such as filtration.

### Compositions

In some embodiments, the present disclosure also provides compositions comprising Compound 1 Fumarate Form E.

In some embodiments, a provided composition comprising Compound 1 Fumarate Form E is substantially free of impurities. As used herein, the term “substantially free of impurities” means that the composition contains no significant amount of extraneous matter. Such extraneous matter may include starting materials, residual solvents, or any other impurities that may result from the preparation of and/or isolation of a crystalline solid form. In some embodiments, the composition comprises at least about 90% by weight of Compound 1 Fumarate Form E. In some embodiments, the composition comprises at least about 95% by weight of Compound 1 Fumarate Form E. In some embodiments, the composition comprises at least about 99% by weight of Compound 1 Fumarate Form E.

In some embodiments, a provided composition comprising Compound 1 Fumarate Form E is substantially pure (e.g., comprises at least about 95%, 97%, 97.5%, 98,% 98.5%, 99%, 99.5%, or 99.8% by weight of the crystalline solid form based on the total weight of the composition). In some embodiments, a composition comprising Compound 1 Fumarate Form E comprises no more than about 5.0 percent of total organic impurities. In some embodiments, a composition comprising Compound 1 Fumarate Form E comprises no more than about 3.0 percent of total organic impurities. In some embodiments, a composition comprising Compound 1 Fumarate Form E comprises no more than about 1.5 percent of total organic impurities. In some embodiments, a composition comprising Compound 1 Fumarate Form E comprises no more than about 1.0 percent of total organic impurities. In some embodiments, a composition comprising Compound 1 Fumarate Form E comprises no more than about 0.5 percent of total organic impurities. In some embodiments, the percent of total organic impurities is measured by HPLC.

In some embodiments, a composition comprises Compound 1 Fumarate Form E and an amorphous solid form (e.g., an amorphous solid form of Compound 1 and/or Compound 1 Fumarate). In some embodiments, a composition comprising a crystalline solid form is substantially free of an amorphous solid form. As used herein, the term “substantially free of an amorphous solid form” means that the composition contains no significant amount of an amorphous solid form. In some embodiments, the composition comprises at least about 90% by weight of Compound 1 Fumarate Form E. In some embodiments, the composition comprises at least about 95% by weight of Compound 1 Fumarate Form E. In some embodiments, the composition comprises at least about 99% by weight of Compound 1 Fumarate Form E. In some embodiments, the composition comprises no more than about 10% by weight of an amorphous solid form (e.g., an amorphous solid form of Compound 1 and/or Compound 1 Fumarate). In some embodiments, the composition comprises no more than about 5% by weight of an amorphous solid form (e.g., a crystalline solid form of Compound 1 and/or Compound 1 Fumarate). In some embodiments, the composition comprises no more than about 1% by weight of an amorphous solid form (e.g., a crystalline solid form of Compound 1 and/or Compound 1 Fumarate).

### Pharmaceutical Compositions

In some embodiments, the present disclosure provides a pharmaceutical composition comprising Compound 1 Fumarate Form E and a pharmaceutically acceptable carrier.

In some embodiments, provided pharmaceutical compositions comprise an amount of Compound 1 (e.g., in the form of Compound 1 Fumarate Form E) that is effective to measurably inhibit estrogen receptor (ER) or a mutant thereof in a biological sample or patient. In some embodiments, provided pharmaceutical compositions are formulated for oral administration.

In some embodiments, provided pharmaceutical compositions comprise Compound 1 Fumarate Form E and one or more fillers, disintegrants, lubricants, glidants, anti-adherents, and/or anti-statics, etc.

Pharmaceutical compositions of the present disclosure may be administered orally, parenterally, by inhalation spray, topically, rectally, nasally, buccally, vaginally, intraperitoneally, intracisternally or via an implanted reservoir. In some embodiments, provided pharmaceutical compositions are administered orally, intraperitoneally or intravenously. In some embodiments, provided pharmaceutical compositions are administered orally.

In some embodiments, a provided pharmaceutical composition is an oral dosage form (e.g., a capsule or a tablet). In some embodiments, a provided pharmaceutical composition is a tablet. In some embodiments, a provided pharmaceutical composition is a capsule.

In some embodiments, a provided pharmaceutical composition is a solid pharmaceutical composition (e.g., a solid dosage form such as a capsule or tablet).

In some embodiments, a provided pharmaceutical composition comprises an amount of Compound 1 suitable to provide a human with a dose of Compound 1 that corresponds to 3, 4, 5, 6, 7, 8, 9, 10, 11, 12, 13, 14, 15, 16, 17, 18, 19, 20, 21, 22, 23, 24, 25, 26, 27, 28, 29, or 30 mg/kg in a mouse. In some embodiments, a provided pharmaceutical composition comprises an amount of Compound 1 suitable to provide a human with a dose of Compound 1 that corresponds to at least 3 mg/kg in a mouse. In some embodiments, a provided pharmaceutical composition comprises an amount of Compound 1 suitable to provide a human with a dose of Compound 1 that corresponds to at least 5 mg/kg in a mouse. In some embodiments, a provided pharmaceutical composition comprises an amount of Compound 1 suitable to provide a human with a dose of Compound 1 that corresponds to at least 10 mg/kg in a mouse. In some embodiments, a provided pharmaceutical composition comprises an amount of Compound 1 suitable to provide a human with a dose of Compound 1 that corresponds to at least 15 mg/kg in a mouse. In some embodiments, a provided pharmaceutical composition comprises an amount of Compound 1 suitable to provide a human with a dose of Compound 1 that corresponds to at least 20 mg/kg in a mouse. In some embodiments, a provided pharmaceutical composition comprises an amount of Compound 1 suitable to provide a human with a dose of Compound 1 that corresponds to at least 25 mg/kg in a mouse. In some embodiments, a provided pharmaceutical composition comprises an amount of Compound 1 suitable to provide a human with a dose of Compound 1 that corresponds to at least 30 mg/kg in a mouse.

In some embodiments, a provided pharmaceutical composition is administered once daily (QD). In some embodiments, a provided pharmaceutical composition is administered twice daily (BID). In some embodiments, a provided pharmaceutical composition is administered every other day (QOD). In some embodiments, a provided pharmaceutical composition is administered once weekly (QW). In some embodiments, a provided pharmaceutical composition is administered once every four weeks (Q4W).

In some embodiments, a provided pharmaceutical composition (e.g., a unit dosage form) comprises about 15 mg to about 120 mg of Compound 1. In some embodiments, a provided pharmaceutical composition (e.g., a unit dosage form) comprises about 15 mg to about 100 mg of Compound 1. In some embodiments, a provided pharmaceutical composition (e.g., a unit dosage form) comprises about 60 mg to about 120 mg of Compound 1. In some embodiments, a provided pharmaceutical composition (e.g., a unit dosage form) comprises about 15 mg, about 20 mg, about 30 mg, about 40 mg, about 50 mg, about 60 mg, about 70 mg, about 80 mg, about 90 mg, or about 100 mg of Compound 1. In some embodiments, a provided pharmaceutical composition (e.g., a unit dosage form) comprises about 15 mg of Compound 1. In some embodiments, a provided pharmaceutical composition (e.g., a unit dosage form) comprises about 30 mg of Compound 1. In some embodiments, a provided pharmaceutical composition (e.g., a unit dosage form) comprises about 60 mg of Compound 1. In some embodiments, a provided pharmaceutical composition (e.g., a unit dosage form) comprises about 90 mg of Compound 1. In some embodiments, a provided pharmaceutical composition (e.g., a unit dosage form) comprises about 120 mg of Compound 1. In some embodiments, a unit dosage form is a capsule. In some embodiments, a unit dosage form is a tablet.

It will be appreciated that reference to an amount (e.g., in mg) of Compound 1 in relation to, e.g., a pharmaceutical composition, dosing regimen, etc., means the amount of Compound 1 in free base form. Accordingly, Compound 1 may be provided and/or utilized as, e.g., a salt form, such that the amount of the salt (or other form) is an amount that corresponds to the “free base equivalent” of Compound 1.

In some embodiments, a provided pharmaceutical composition is prepared by (i) providing Compound 1 Fumarate Form E; and (ii) formulating the Compound 1 Fumarate Form E with suitable excipients, to provide the pharmaceutical composition.

### Uses

Compounds and compositions described herein are generally useful for the inhibition of the estrogen receptor (ER) and mutants thereof. In some embodiments, the present disclosure encompasses the insight that compounds and compositions described herein are useful for treatment of an ER-associated disorder (e.g., an ER-associated cancer, such as breast cancer, including metastatic brain cancer), detection of the same, and/or characterization of certain tumors.

For example, in some embodiments, the present disclosure provides certain methods of treatment in a subject having an ER-associated disease, disorder, or condition. In some embodiments, an ER-associated disease, disorder or condition is a cancer. In some embodiments, an ER-associated disease, disorder or condition is selected from breast cancer, bone cancer, lung cancer, colorectal cancer, endometrial cancer, prostate cancer, ovarian cancer, vaginal cancer, endometriosis, and uterine cancer. In some embodiments, an ER-associated disease, disorder, or condition is breast cancer.

In some embodiments, a subject has been determined or is suspected of having a cancer that has metastasized (e.g., to the brain, bones, lungs, liver, or the central nervous system). In some embodiments, a subject has been determined or is suspected of having brain metastases. In some embodiments, the subject has developed brain metastases related to an ER-associated cancer, e.g., breast cancer, or a mutation to the estrogen receptor.

In some embodiments, a provided method comprises administering Compound 1 (e.g., as Compound 1 Fumarate Form E) to a subject previously treated with an ER inhibitor. In some such embodiments, a provided method comprises administering Compound 1 (e.g., as Compound 1 Fumarate Form E) to a subject previously treated with a Selective Estrogen Receptor Modulator (SERM), including, for example, tamoxifen, endoxifene, raloxifene, toremifene, lasofoxifene, and ospemifene.

In some embodiments, a provided method comprises administering Compound 1, or a crystalline form or complex form thereof, to a subject suffering from an ER-associated disorder (e.g., breast cancer) that is unresponsive to therapy with a SERM, including, for example, tamoxifen, endoxifene, raloxifene, toremifene, lasofoxifene, and ospemifene.

In some embodiments, a subject has relapsed during or following therapy with a SERM, including, for example, tamoxifen, endoxifene, raloxifene, toremifene, lasofoxifene, and ospemifene.

In some embodiments, a provided method comprises administering Compound 1 (e.g., as Compound 1 Fumarate Form E) to a subject with estrogen receptor positive (ER+) and human epidermal growth factor receptor negative (HER−) disease. In some embodiments, a provided method comprises administering Compound 1 (e.g., as Compound 1 Fumarate Form E) to a subject with estrogen receptor positive (ER+) and human epidermal growth factor receptor positive (HER+) disease.

In some embodiments, Compound 1 (e.g., as Compound 1 Fumarate Form E) is administered to the subject in an amount that is from about to 15 mg to about 360 mg. In some embodiments, Compound 1 is administered to the subject in an amount that is from about to 30 mg to about 360 mg. In some embodiments, Compound 1 is administered to the subject in an amount that is from about to 30 mg to about 300 mg. In some embodiments, Compound 1 is administered to the subject in an amount that is from about to 60 mg to about 120 mg. In some embodiments, Compound 1 is administered to the subject in an amount that is from about 15 mg to about 100 mg. In some embodiments, Compound 1 is administered to the subject in an amount that is about 15 mg, about 20 mg, about 30 mg, about 40 mg, about 50 mg, about 60 mg, about 70 mg, about 80 mg, about 90 mg, or about 100 mg. In some embodiments, Compound 1 is administered to the subject in an amount that is about 120 mg, about 150 mg, about 210 mg, or about 300 mg. In some embodiments, Compound 1 is administered to the subject in an amount that is about 30 mg. In some embodiments, Compound 1 is administered to the subject in an amount that is about 60 mg. In some embodiments, Compound 1 is administered to the subject in an amount that is about 90 mg. In some embodiments, Compound 1 is administered to the subject in an amount that is about 120 mg.

In some embodiments, Compound 1 (e.g., as Compound 1 Fumarate Form E) is administered to the subject in an amount that is about 15 mg to about 360 mg per day (QD). In some embodiments, Compound 1 is administered to the subject in an amount that is about 30 mg to about 360 mg per day (QD). In some embodiments, Compound 1 is administered to the subject in an amount that is about 30 mg to about 300 mg per day (QD). In some embodiments, Compound 1 is administered to the subject in an amount that is about 60 mg to about 120 mg per day (QD). In some embodiments, Compound 1 is administered to the subject in an amount that is from about 15 mg to about 100 mg QD. In some embodiments, Compound 1 is administered to the subject in an amount that is about 15 mg, about 20 mg, about 30 mg, about 40 mg, about 50 mg, about 60 mg, about 70 mg, about 80 mg, about 90 mg, or about 100 mg QD. In some embodiments, Compound 1 is administered to the subject in an amount that is about 120 mg, about 150 mg, about 210 mg, or about 300 mg QD. In some embodiments, Compound 1 is administered to the subject in an amount that is about 30 mg QD. In some embodiments, Compound 1 is administered to the subject in an amount that is about 60 mg QD. In some embodiments, Compound 1 is administered to the subject in an amount that is about 90 mg QD. In some embodiments, Compound 1 is administered to the subject in an amount that is about 120 mg QD.

In some embodiments, Compound 1 (e.g., as Compound 1 Fumarate Form E) is administered to the subject in a unit dosage form. In some embodiments, unit dosage form is a capsule or tablet. In some embodiments, a unit dosage form comprises about 15 mg to about 120 mg of Compound 1. In some embodiments, a unit dosage form comprises about 15 mg to about 100 mg of Compound 1. In some embodiments, a unit dosage form comprises about 60 mg to about 120 mg of Compound 1. In some embodiments, a unit dosage form comprises about 15 mg, about 20 mg, about 30 mg, about 40 mg, about 50 mg, about 60 mg, about 70 mg, about 80 mg, about 90 mg, or about 100 mg of Compound 1. In some embodiments, a unit dosage form comprises about 15 mg of Compound 1. In some embodiments, a unit dosage form comprises about 30 mg of Compound 1. In some embodiments, a unit dosage form comprises about 60 mg of Compound 1. In some embodiments, a unit dosage form comprises about 90 mg of Compound 1. In some embodiments, a unit dosage form comprises about 120 mg of Compound 1. In some embodiments, a unit dosage form is a capsule. In some embodiments, a unit dosage form is a tablet.

In some embodiments, a total daily dose of Compound 1 administered to the subject is in an amount that is about 15 mg to about 360 mg per day (QD). In some embodiments, a total daily dose of Compound 1 administered to the subject is about 30 mg to about 360 mg. In some embodiments, a total daily dose of Compound 1 administered to the subject is about 30 mg to about 300 mg. In some embodiments, a total daily dose of Compound 1 administered to the subject is about 60 mg to about 120 mg. In some embodiments, a total daily dose of Compound 1 administered to the subject is in an amount that is from about 15 mg to about 100 mg QD. In some embodiments, a total daily dose of Compound 1 administered to the subject is in an amount that is about 15 mg, about 20 mg, about 30 mg, about 40 mg, about 50 mg, about 60 mg, about 70 mg, about 80 mg, about 90 mg, or about 100 mg QD. In some embodiments, a total daily dose of Compound 1 administered to the subject is about 120 mg, about 150 mg, about 210 mg, or about 300 mg. In some embodiments, a total daily dose of Compound 1 administered to the subject is in an amount that is about 30 mg QD. In some embodiments, a total daily dose of Compound 1 administered to the subject is about 60 mg. In some embodiments, a total daily dose of Compound 1 administered to the subject is about 90 mg. In some embodiments, a total daily dose of Compound 1 administered to the subject is about 120 mg.

### Combination Therapy

The present disclosure encompasses the recognition that a combination of certain agents can beneficially be used to completely antagonize the estrogen receptor. Accordingly, in some embodiments, the present disclosure provides a method of treating a subject suffering from an ER-associated disorder (e.g., a cancer, e.g., a breast cancer) comprising administering a complete estrogen receptor antagonist and a secondary anti-cancer agent. For example, in some embodiments, a complete estrogen receptor antagonist is Compound 1 (e.g., as Compound 1 Fumarate Form E). In some embodiments, a secondary anti-cancer agent is a CDK 4/6 inhibitor, a PI3KCA inhibitor, or an mTOR inhibitor.

In some embodiments, the present disclosure provides a method of treating a patient or subject suffering from a cancer, the method comprising administering a complete estrogen receptor antagonist (e.g., Compound 1, e.g., as Compound 1 Fumarate Form E) and a CDK4/6 inhibitor (i.e., an agent that inhibits one or both of CDK4 and CDK6). In some embodiments, an anti-cancer agent is a CDK4/6 inhibitor selected from palbociclib, ribociclib, abemaciclib, lerociclib, trilaciclib, and SHR6390. In some embodiments, a CDK4/6 inhibitor is palbociclib. In some embodiments, a CDK4/6 inhibitor is ribociclib. In some embodiments, a CDK4/6 inhibitor is abemaciclib. In some embodiments, a CDK4/6 inhibitor is lerociclib. In some embodiments, a CDK4/6 inhibitor is trilaciclib. In some embodiments, a CDK 4/6 inhibitor is SHR6390.

In some embodiments, the present disclosure provides a method of treating a patient or subject suffering from a cancer, the method comprising administering a complete estrogen receptor antagonist (e.g., Compound 1, e.g., as Compound 1 Fumarate Form E) and a PIK3CA inhibitor. In some embodiments, a PIK3CA inhibitor is selected from alpelisib, taselisib, and LY3023414. In some embodiments, a PIK3CA inhibitor is alpelisib. In some embodiments, a PIK3CA inhibitor is taselisib. In some embodiments, a PIK3CA inhibitor is LY3023414.

In some embodiments, the present disclosure provides a method of treating a patient or subject suffering from a cancer, the method comprising administering a complete estrogen receptor antagonist (e.g., Compound 1, e.g., as Compound 1 Fumarate Form E) and an mTOR inhibitor. In some embodiments, an mTOR inhibitor is selected from sirolimus, temsirolimus, everolimus, and LY3023414. In some embodiments, an mTOR inhibitor is sirolimus. In some embodiments, an mTOR inhibitor is temsirolimus. In some embodiments, an mTOR inhibitor is everolimus. In some embodiments, an mTOR inhibitor is LY3023414.

In some embodiments, the present disclosure provides methods of treating a subject with ER+ and HER+ disease with a complete estrogen receptor antagonist ((e.g., Compound 1, e.g., as Compound 1 Fumarate Form E) and a HER2 inhibitor. In some embodiments, a HER2 inhibitor is selected from tucatinib, pertuzumab, lapatinib, trastuzumab, ado-trastuzumab emtansine, trastuzumab deruxtecan, and neratinib.

It is understood that combination therapy comprising a complete estrogen receptor antagonist and an anti-cancer agent described herein can comprise administration of the agents simultaneously or separately. For example, in some embodiments, a complete estrogen receptor antagonist and an anti-cancer agent are administered simultaneously. In some embodiments, an anti-cancer agent is administered prior to administration of a complete estrogen receptor antagonist. In some embodiments, an anti-cancer agent is administered after administration of a complete estrogen receptor antagonist.

### EXAMPLES

The Examples provided herein document and support certain aspects of the present disclosure but are not intended to limit the scope of any claim. The following non-limiting examples are provided to further illustrate certain teachings provided by the present disclosure. Those of skill in the art, in light of the present application, will appreciate that various changes can be made in the specific embodiments that are illustrated in the present Examples without departing from the spirit and scope of the present teachings.

The following abbreviations may be used in the Examples below: aq. (aqueous); ACN (acetonitrile); CSA (camphorsulfonic acid); d (day or days); DCM (dichloromethane); DEA (diethylamine); DHP (dihydropyran); DMF (N,N-dimethylformamide); DIPEA (N,N-diisopropylethylamine); DMAP (4-dimethylaminopyridine); DMSO (dimethyl sulfoxide); EA (ethyl acetate); ee (enantiomeric excess); equiv. (equivalent); ethanol (EtOH); h (hour or hours); Hex (hexanes); HPLC (high-performance liquid chromatography); IPA (isopropyl alcohol); KHMDS (potassium bis(trimethylsilyl)amide); LAH (lithium aluminum hydride); LCMS (liquid chromatography-mass spectrometry); LDA (lithium diisopropylamide); LiHMDS (lithium bis(trimethylsilyl)amide); MeOH (methanol); min (minute or minutes); NMR (nuclear magnetic resonance); Pd/C (palladium on carbon); PPh30 (triphenylphosphine oxide); Pt/C (platinum on carbon); rb (round-bottomcd); Rf (retention factor); rt or RT (room temperaturc); SM (starting material); TEA (triethylamine); THF (tetrahydrofuran); THP (tetrahydropyran); TLC (thin layer chromatography); TsOH (p-toluenesulfonic acid or tosylic acid); and UV (ultraviolet).

### Materials & Methods

### X-Ray Powder Diffraction (XRPD)

XRPD was performed with PANalytical X'Pert PRO MPD or Empyrean diffractometers using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640f) was analyzed to verify the observed position of the Si 111 peak was consistent with the NIST-certified position. A specimen of the sample was sandwiched between Kapton films and analyzed in transmission geometry. A beam-stop, short antiscatter extension, and an antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 5.5.

Alternatively, XRPD was performed using a Bruker D8 Focus X-ray diffractometer equipped with LynxEye detector. Samples were scanned from 3° to 42° (2θ), at a step size of 0.02° (20). The tube voltage and current were 40 KV and 40 mA, respectively.

### Thermogravimetric Analysis (TGA) and Differential Scanning Calorimetry (DSC)

TGA/DSC analyses were performed using a Mettler-Toledo TGA/DSC3+ analyzer. Temperature and enthalpy adjustments were performed using indium, tin, zinc, aluminum, gold, and phenyl salicylate, and then verified with indium. The balance was verified with calcium oxalate. The samples were placed in an open aluminum pan, hermetically sealed, the lid pierced, and then inserted into the TG furnace. A weighed aluminum pan configured as the sample pan was placed on the reference platform. The furnace was heated under nitrogen.

Alternatively, TGA was performed using a TGA Q500 (TA Instruments, US). About 1-5 mg of sample was placed in an open tarred aluminum pan, automatically weighed, and inserted into the TGA furnace. The sample was heated at a rate of 10° C./min to the final temperature (about 300° C.). DSC characterization was conducted on a DSC 250 (TA Instruments, US). About 1-5 mg of sample was placed into a DSC pinhole pan. The sample was heated at a rate of 10° C./min to the final temperature (about 300° C.). The change of heat flux with temperature was recorded.

### Dynamic Vapor Sorption (DVS)

DVS was performed using Intrinsic DVS (System Measurement System, UK). About 30-50 mg of sample was placed in a sample basked and hung in the measuring chamber. For an isotherm test, the chamber temperature was maintained by a water bath at a constant 25±1° C. The sample was tested at a targeted RH from 0 to 90% full cycle in step mode. The analysis was performed in 10% RH increments. Time duration at each RH was set as 60 min so that the sample could reach equilibrium with the chamber environment. Data were collected in 20 s increments.

### Gas Chromatography (GC)

GC analysis was performed on GC8890 (Agilent, US), using helium gas as carrier gas and nitrogen gas as makeup gas with a FID detector. The sample was 10 mg/mL in dimethylacetamide. The vaporized sample was carried by the carrier gas (mobile phase) into the chromatographic column. The parameters are summarized below:

| | |
|---|---|
| Injector | Auto-sampler, fast plunger mode, direct |
| | injection |
| Carrier gas | Helium |
| Chromatographic | DB-64, Agilent, 30 m × 0.53 mm × 3 mm |
| column | |
| Column flow | 6.4 mL/min |
| Inlet | Inlet temperature is 250° C. |
| | Split ratio is 10:1 |
| | Constant pressure mode |
| Temp. Program | 40° C. for 5 min; ramp at 10° C./min to 220° C. |
| Detector | Detector temperature is 250° C. |
| | Used nitrogen as makeup gas |
| | H₂:Air:N₂ = 40:400:30 (mL/min) |
| Injection volume | 1 μL |
| Needle wash | Used diluents as needle washing solvent |

### Example 1: Synthesis of Compound 1

A complete synthesis of Compound 1 is provided in PCT Pub. No. WO 2017/059139, which is incorporated herein by reference and repeated below.

### Preparation of 4-((1-propylazetidin-3-yl)oxy)benzaldehyde

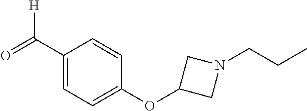 [SMILES: CCCN1CC(Oc2ccc(C=O)cc2)C1]

### Step 1: Preparation of 1-propionylazetidin-3-one

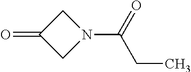

The compound 3-azetidinone hydrochloride (10.000 g, 93.0 mmol, 1.0 equiv.), anhydrous 1,2-dichloroethane (200 mL) and diisopropylethylamine (38.9 mL, 223 mmol, 2.4 equiv.) were added to a round bottom flask (500 mL) to provide a light yellow suspension. The suspension was sonicated for 1 h and then cooled to −10° C. (dry-ice/MeOH) for 10 min. Propionyl chloride (9.8 mL, 112 mmol, 1.2 equiv.) was added dropwise to the cooled suspension to provide an orange solution. The reaction was removed from the bath and stirred at room temperature for 16 h. The solvent was removed to provide a semi-solid. The semi-solid was suspended into EA (300 mL) and the suspension was filtered. The solid was rinsed with EA (2×100 mL). TLC analysis (10% MeOH/DCM, KMnO₇ stain/Heat) indicated there were three spots: Rf: 0.2, 0.5, 0.7. TLC (50% EA/Hex, KMnO₇ stain/Heat) indicated there were two spots: Rf: 1, 0.3. The filtrate was concentrated, adsorbed onto silica gel (25 g) and chromatographed through silica gel (100 g cartridge) with DCM (5 min) then 0-10% MeOH over 15 min. The product came off early from the column in DCM and continued to elute from the column with up to 10% MeOH. TLC in both solvent systems was carried out to determine if any propionyl chloride was present in early fractions. Fractions containing product were pooled and concentrated to afford the title compound as a yellow liquid (11.610 g, 98.2%).

¹H NMR (300 MHZ, CDCl₃) δ: 4.80 (d, J=5.6 Hz, 4H), 2.29 (q, J=7.5 Hz, 2H), 2.01 (s, 3H), 1.18 (t, J=7.5 Hz, 3H).

### Step 2. Preparation of 1-propylazetidin-3-ol

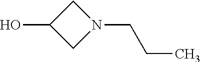

Lithium aluminum hydride (10.397 g, 273.9 mmol, 3.0 equiv.) was suspended into THF (200 mL) and cooled in an ice bath. A solution of 1-propionylazetidin-3-one (11.610 g, 91.3 mmol, 1.0 equiv.) in THF (100 mL) was added dropwise to the reaction mixture via a pressure equalizing addition funnel over 30 min. The addition funnel was removed. The flask was then fitted with a condenser and the reaction was heated at reflux in an oil bath at 75° C. for 16 h. The reaction was cooled in an ice bath for 20 min and sodium sulfate decahydrate (Glauber's salt, 25 g) was added in small portions over 20 min. After complete addition, the mixture was stirred at room temperature for 2 h. The mixture was filtered through a bed of Celite® (2 cm) and the solids rinsed with EA (2×250 mL). The clear solution was concentrated to a pale yellow liquid (9.580 g, 91.1%). NMR indicated the presence of THF and EA. This material was used without further purification in the preparation of the compounds of the examples below.

¹H NMR (300 MHz, CDCl₃) δ: 4.39 (pent, J=6 Hz, 1H), 3.62-3.56 (m, 2H), 2.90-2.85 (m, 2H), 2.41 (t, J=7.5 Hz, 2H), 1.34 (hextet, J=7.2 Hz, 2H), 0.87 (t, J=7.8 Hz, 3H).

### Step 3. Preparation of 4-((1-propylazetidin-3-yl)oxy)benzaldehyde

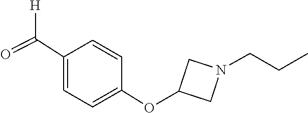 [SMILES: CCCN1CC(Oc2ccc(C=O)cc2)C1]

4-Fluorobenzaldehyde (15.00 g, 120.9 mmol, 0.9 equiv.), 1-propylazetidine-3-ol (15.00 g, 130.2 mmol, 1.0 equiv.), cesium carbonate (88.40 g, 271.3 mmol, 2.1 equiv.) and N,N-dimethylformamide (284 mL) were mixed together with a Teflon™ stir bar in a 500 mL round bottomed flask. The flask was sealed and heated in a heat block at 95° C. for 6 h. The reaction was analyzed by LCMS to indicate the aldehyde was consumed. The suspension was filtered through a sintered glass funnel and the solid was washed with ethyl acetate (100 mL). The filtrate was concentrated to an orange suspension. The suspension was mixed with water (200 mL) and ethyl acetate (200 mL) and the organic layer was washed with water (3×200 mL), brine, dried over anhydrous magnesium sulfate, filtered and concentrated to an orange liquid (21.74 g, 76.1%). The material was used without further purification.

¹HNMR (300 MHz, CDCl₃), δ 9.87 (s, 1H), 7.82 (d, J=9.0 Hz, 2H), 6.86 (d, J=8.7 Hz, 2H), 4.86 (quintet, J=5.7 Hz, 1H), 3.85-3.80 (m, 2H), 3.13-3.08 (m, 2H), 2.48 (t, J=7.2 Hz, 2H), 1.46-1.34 (m, 2H), 0.91 (t, J=7.2 Hz, 3H).

### Preparation of (R)-1-(1H-indol-3-yl)-N—((R)-1-phenylethyl) propan-2-amine

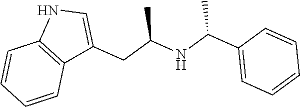 [SMILES: C[C@H](Cc1c[nH]c2ccccc12)N[C@H](C)c1ccccc1]

Indole-3-acetone (25.0 g, 144 mmol, 1.0 equiv.) was added to a solution of (R)-(+)-1-phenylethylamine (23.0 mL, 181 mmol, 1.3 equiv.) in dichloromethane (600 mL) under N₂ at 25° C. and the mixture was allowed to stir for 1 hr. The reaction was cooled to 0-5° C. and sodium triacetoxyborohydride (100 g, 472 mmol, 3.3 equiv.) was added over 30 minutes via powder addition funnel to the ice cooled solution. The orange solution was stirred for 1 h at 0° C. and then was allowed to warm to RT. The reaction was stirred at RT for 19 h. At this time, ESI+ indicated that no indole starting material was present. Saturated NaHCO₃ solution (100 mL) was added in 5 mL portions over 15 min at 10° C. with vigorous stirring. The solution was stirred for 15 min and sat. Na₂CO₃ solution (200 mL) was added over 15 minutes. Solid K₂CO₃ (9 g) was added in 3 g portions at which point the aqueous layer was pH 12 and bubbles had stopped forming. The layers were filtered and separated. The red organic layer was washed with sat. aq. NaHCO₃ (2×100 mL). The aqueous layers were combined and extracted with DCM (2×100 mL). The combined organic layers were dried over Na₂SO₄, filtered and concentrated to give the crude product (49 g). TLC (90:10 DCM: MeOH) showed four spots (Rf=0.63, 0.50, 0.16, 0.26), two of which were the separated diastereomeric major products (Rf=0.16 and 0.26). The crude was adsorbed onto silica gel and purified via flash chromatography (330 g cartridge, 0-100% EA:Hex). Fractions containing the R,R diastereomer were pooled and purified a second time with the same flash chromatography conditions to afford 24 g of product (~82% ee). Previous successful separation was achieved by a silica gel: crude ratio of 40:1, so the mixture was divided into 3 portions and separated on 3×330 g silica gel cartridges (0-40% EA/Hex for 20 min, isocratic 40% EA/Hex 40 min). All fractions containing the desired product were >99% diastereomerically pure. Pure fractions were concentrated and pooled to yield (R)-1-(1H-indol-3-yl)-N—((R)-1-phenylethyl)-propan-2-amine as an orange semi-solid (11.91 g, 29.6%).

¹H NMR (CDCl₃, 300 MHz) R,R diastereomer: δ 0.96 (d, J=6.6 Hz, 3H), 1.30 (d, J=6.6 Hz, 3H), 2.68 (q, J=7.2 Hz, 1H), 2.97 (m, 2H) 4.00 (q, J=6.3 Hz, 1H), 7.43-6.97 (m, 10H), 7.96 (br s, 1H). R,S diastereomer: δ 1.11 (d, J=5.7 Hz, 3H), 1.30 (d, J=5.4 Hz, 3H) 2.80 (m, 3H), 3.92 (q, J=6.9 Hz, 1H), 6.93-7.40 (m, 10H), 8.13 (br s, 1H); the aromatic region was difficult to distinguish from the R,R diastereomer due to lack of purity.

LCMS: ES+ [M+H]+ 279.0.

### Preparation of (2R)-1-(1H-indol-3-yl) propan-2-amine

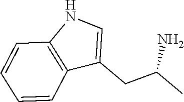

The compound (R)-1-(1H-indol-3-yl)-N—((R)-1-phenylethyl) propan-2-amine (11.91 g, 42.8 mmol, 1.0 equiv.) was dissolved in methanol (250 mL) and added to a 2 L Parr bottle and the solution was sparged with N₂ for 10 min. 20% Pd(OH)₂ on carbon wet with water (10.71 g, 76.3 mmol, 1.8 equiv.) was added and the bottle was pressurized with 50 psi of hydrogen and shaken in a Parr apparatus for 22 h, LCMS analysis indicated that the reaction was completed. The suspension was filtered through Celite® and concentrated to remove MeOH. The crude was dissolved into DCM and washed with saturated Na₂CO₃ solution (50 mL) and the aqueous layer was extracted with DCM (2×50 mL). The organic layers were combined, dried, and concentrated to yield (2R)-1-(1H-indol-3-yl) propan-2-amine as a light brown solid that did not require further purification (6.68 g, 89.6%).

¹H NMR (CDCl₃, 300 MHz) δ 1.17 (d, J=6.6 Hz, 3H), 2.66 (dd, J=8.4, 14.7 Hz, 1H), 2.88 (dd, J=5.4, 14.1 Hz, 1H), 3.27 (sextet, J=1.5 Hz, 1H), 7.05-7.22 (m, 3H), 7.37 (d, J=7.5 Hz, 1H), 7.62 (d, J=8.7 Hz, 1H), 8.00 (br s, 1H).

LCMS: ES+ [M+H]+ 174.9.

### Preparation of 2-fluoro-2-methylpropanol

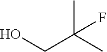

Methyl 2-fluoro-2-methylpropionate (5.01 g, 40.5 mmol, 1.0 equiv.) was added dropwise over 15 min to a stirred suspension of lithium aluminum hydride (2.50 g, 65.9 mmol, 1.6 equiv.) in anhydrous diethyl ether (100 mL) cooled in an ice bath. After 2 hours, 2.0 mL water, 2.0 mL 15% w/v NaOH, and 5.0 mL water were added sequentially dropwise. After 15 min, the white suspension was diluted with DCM, gravity filtered through Celite®, and the solids were washed with DCM. The filtrate was concentrated (200 mbar, 25° C.) to afford 2-fluoro-2-methylpropanol as a colorless oil (2.09 g, 56.1%).

¹H NMR (300 MHz, CDCl₃) δ 1.34 (d, J=21.3 Hz, 6H), 1.95 (br t, 1H), 3.56 (dd, J=6.6, 20.7 Hz, 2H).

### Preparation of 2-fluoro-2-methylpropyl trifluoromethanesulfonate

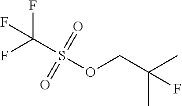

Trifluoromethanesulfonic anhydride (5.0 mL, 29.7 mmol, 1.3 equiv.) was added dropwise to a 0° C. solution of 2-fluoro-2-methylpropanol (2.090 g, 22.7 mmol, 1.0 equiv.) and 2,6-lutidine (3.40 mL, 29.4 mmol, 1.3 equiv.) in DCM (25 mL) over 30 minutes. After 2 hours, the red solution had turned light brown. TLC (20:80 EA:Hex, KMnO₄ stain) indicated that the starting material was not present. The reaction mixture was washed with 1M HCl solution (2×20 mL) and sat. NaHCO₃ solution (2×20 mL). The aqueous layers were each back extracted with DCM (20 mL). The combined organic layers were dried with Na₂SO₄, filtered and concentrated under reduced pressure (150 mbar, 25° C.) to afford 2-fluoro-2-methylpropyl trifluoromethanesulfonate as a red oil (4.39 g, 86.3%).

¹H NMR (300 MHz, CDCl₃) δ 1.46 (d, J=20.4 Hz, 6H), 4.41 (d, J=18.6 Hz, 2H). ¹⁹F NMR (282 MHZ, CDCl₃) δ −147.1, −74.5.

### Preparation of (R)—N-(1-(1H-indol-3-yl) propan-2-yl)-2-fluoro-2-methylpropan-1-amine

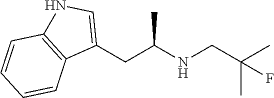 [SMILES: C[C@H](Cc1c[nH]c2ccccc12)NCC(C)(C)F]

The compound 2-fluoro-2-methylpropyl trifluoromethanesulfonate (9.587 g, 42.8 mmol, 1.1 equiv.) (solution in DCM, 16% DCM by wt %, 11.4384 g) was added to a solution of (2R)-1-(1H-indol-3-yl) propan-2-amine (6.680 g, 38.3 mmol, 1.0 equiv.), anhydrous 1,4-dioxanes (60.000 ml, 701.4 mmol, 18.3 equiv.), and freshly-distilled diisopropylethylamine (8.500 ml, 48.8 mmol, 1.3 equiv.). The dark brown solution was heated at 90° C. for 3 hours. After 3 h, LCMS indicated that a small amount of indolamine starting material was still present. TLC (10% MeOH/DCM) indicated triflate (Rf=0.54) had been used up. NMR of unused triflate SM (286-30) indicated the triflate had not decomposed overnight, so another 0.1 equiv (0.9883 g, 13% DCM wt %, 0.8563 g triflate SM) was added and the reaction was heated for 2 h at 90° C. LCMS indicated the reaction had completed and TLC (10% MeOH/DCM) showed one spot (Rf=0.24) (TLC with 50% EA/Hex, 1 streaked spot Rf<=0.12, another spot at Rf=0). EtOAc (50 mL) was added and the solution was washed with NaHCO₃ (2×50 mL) and the combined aqueous layer was washed with EtOAc (50 mL). The combined organic extracts were dried over Na₂SO₄ and concentrated under reduced pressure. The crude (brown oil, 14.8 g) was purified via flash silica chromatography (240 g cartridge, 0-100% EA/Hex). The desired product eluted as a long tailing peak. Pure fractions were concentrated to yield (R)—N-(1-(1H-indol-3-yl) propan-2-yl)-2-fluoro-2-methylpropan-1-amine (4.211 g, 17.0 mmol) as a dark yellow oil.

¹H NMR (300 MHZ, CDCl₃) δ 1.10 (d, J=6.3 Hz, 3H), 1.34 (dd, J=3.0, 21.9 Hz, 6H), 2.68-2.95 (m, 4H), 3.02 (sextet, J=6.6 Hz, 1H), 7.05 (d, J=2.4 Hz, 1H), 7.26-7.11 (m, 2H), 7.36 (d, J=6.9 Hz, 1H), 7.62 (d, J=7.5 Hz, 1H), 8.18 (br s, 1H). ¹⁹F NMR (282 MHz, CDCl₃) δ −144.2. m/z: ES+ [M+H]+ 249.0.

### Preparation of Compound 1

4-((1-propylazetidin-3-yl)oxy)benzaldehyde (0.096 g, 0.4 mmol, 1.3 equiv.) was added to a solution of (R)—N-(1-(1H-indol-3-yl) propan-2-yl)-2-fluoro-2-methylpropan-1-amine (0.070 g, 0.3 mmol, 1.0 equiv.) in anhydrous toluene (1.50 mL) and glacial acetic acid (0.100 mL, 1.7 mmol, 6.2 equiv.). Molecular sieves were added and the solution was stirred under N₂ in the dark at 80° C. for 8 hours. The reaction solution was diluted in DCM, filtered, and washed with saturated Na₂CO₃ solution. The aqueous layer was extracted with DCM and the combined organic layers were dried over Na₂SO₄. The solution was filtered and concentrated. The residue was dissolved into acetonitrile (2 mL) and filtered through a syringe filter before purification via prep LC (40 to 90% ACN:H₂O over 18 min, followed by isocratic 90% ACN for 7 min). Pure fractions were concentrated and dried to afford (1R,3R)-2-(2-fluoro-2-methylpropyl)-3-methyl-1-(4-((1-propylazetidin-3-yl)oxy)phenyl)-2,3,4,9,-tetrahydro-1H-pyrido[3,4-b]indole as a white powder.

¹H NMR (300 MHZ, CDCl₃) δ 0.90 (t, J=7.5 Hz, 3H), 1.09 (d, J=7.2 Hz, 3H), 1.26-1.50 (m, 8H), 2.45-2.77 (m, 6H), 3.01 (t, J=7.2 Hz, 2H), 3.34 (m, 1H), 3.77 (m, 2H), 4.60 (quin, J=5.7 Hz, 1H), 5.03 (s, 1H), 6.64 (d, J=8.1 Hz, 2H), 7.10-7.21 (m, 5H), 7.54 (d, J=7.5 Hz, 1H), 8.19 (br s, 1H). m/z: ES+ [M+H]+ 450.2.

### Example 2: Preparation and Characterization of Compound 1 Fumarate Form E

Compound 1 Fumarate Form E was obtained according to the following exemplary procedure: Compound 1 Fumarate Form A (~30-100 mg) was slurried in isopropanol at ambient temperature for 20 days. Solids were isolated to give Compound 1 Fumarate Form E. Compound 1 Fumarate Form A was prepared as follows: Fumaric acid (52.6 mg) was weighed into a 20-mL glass vial. A 40 mg/mL solution of amorphous Compound 1 in ethyl acetate (15 mL) was added to the vial, and the mixture stirred at RT. A sample collected after 1 day of stirring was confirmed to be Compound 1 Fumarate Form A with XRPD. The resulting suspension was filtered, and the wet cake dried at 50° C. for 5 h under vacuum. Solids were collected to give Compound 1 Fumarate Form A (231.9 mg, ~92.2% yield).

The XRPD pattern of Compound 1 Fumarate Form E is shown in FIG. 1.

As shown by DSC curve in FIG. 2, the sample displayed one endothermic peak at 147° C. (onset)/156° C. (peak). FIG. 2 also shows the TGA curve, which shows no weight loss up to 180° C. Compound 1 Fumarate Form E was determined to be unsolvated.

Analysis by ¹HNMR indicated a ~1:1 stoichiometry of fumaric acid:Compound 1.

Compound 1 Fumarate Form E was also prepared as follows: Amorphous Compound 1 (80.5 mg) and fumaric acid (24.4 mg) were mixed and suspended in isopropanol (2 mL) with stirring on a magnetic stirrer. Heptane (1 mL) was added to the clear solution, and the sample was placed in the freezer. After approximately one day, solids were isolated via centrifugation with filtration and analyzed by XRPD.

Compound 1 Fumarate Form E was also prepared as follows: Amorphous Compound 1 (2.0 g) and isopropanol (30 mL) were charged into a 50 mL reactor vessel. Compound 1 dissolved under 40° C., and the agitation rate was kept at 300 rpm (two-blade paddle). Fumaric acid (0.5 equiv) was added and dissolved after stirring for 5 min. Seeds of Compound 1 Fumarate Form E (1.0 wt %) were then added. After stirring for 1 h, fumaric acid (0.2 equiv) was added. After stirring for another 1 h, fumaric acid (0.2 equiv) was added. After stirring for another 1 h, fumaric acid (0.3 equiv) was added. Then, heptane (30 mL) was added within 4 h. The mixture was kept at 40° C. for 1 h, then cooled to 25° C. within 3 h, and then stirred for 10 h. The suspension was filtered, and the wet cake dried at 40° C. for 16 h in a vacuum oven to give Compound 1 Fumarate Form E (2.2 g, 90% yield).

Compound 1 Fumarate Form E was also prepared as follows: Amorphous Compound 1 (20.0 g) and isopropanol (300 mL) were charged into a 1000 mL reactor vessel. Compound 1 dissolved under 40° C., and the agitation rate was kept at 300 rpm (retreat curve impeller, RCI). Fumaric acid (0.5 equiv) was added and stirred for 20 min. Seeds of Compound 1 Fumarate Form E (1.0 wt %) were then added. After stirring for 1 h, fumaric acid (0.2 equiv) was added slowly. After stirring for another 1 h, fumaric acid (0.2 equiv) was added slowly. After stirring for another 1 h, fumaric acid (0.3 equiv) was added slowly. Then, heptane (300 mL) was added within 4 h. The mixture was kept at 40° C. for 1 h, then cooled to 25° C. within 3 h, and then stirred for 10 h. The suspension was filtered, and the wet cake dried at 40° C. for 16 h in a vacuum oven to give Compound 1 Fumarate Form E (23.2 g, 92% yield). Analysis by XRPD (FIG. 3) confirmed that the material was Compound 1 Fumarate Form E. TGA analysis (FIG. 4) showed a weight loss of 0.438% starting at 151.2° C., and DSC (FIG. 5) showed a melting point of 157.9° C. (peak temperature). Gas chromatography (GC) indicated that isopropanol was present at 3264 ppm and heptane was present at 1434 ppm. DVS analysis (FIG. 6) indicated that Compound 1 Fumarate Form E was not hygroscopic.

Exemplary XRPD data of Compound 1 Fumarate Form E are summarized below:

Position (degrees 2-Theta)

5.827
7.025
8.687
10.905
12.881
13.425
14.675
15.649
16.084
16.654
17.716
18.455
18.881
19.632
19.990
21.733
22.003
22.308
23.834
24.626
25.024
26.481
27.373
28.598
29.324
29.812
30.046
35.685
39.698
40.315
40.969

### Example 3: Solubility Studies

Solubility studies of Compound 1 and Compound 1 Fumarate in different solvents at 25° C. were performed. Solubility was measured by the dynamic method and gravimetric method.

Dynamic method: Under the condition of a certain amount of solute and certain temperature, solvent was gradually added with stirring for 15 min to reach equilibrium. When solute is completely dissolved, amount of solvent was recorded and the solubility calculated.

Gravimetric method: Excess solids and a certain amount of solvent were added 8 mL vials, stirred for 24 hours, and filtered. 1 mL of clear upper layer of liquid was taken, dried at 50° C. for 24 h, weighed, and the solubility calculated.

Compound 1 Fumarate Form A Anhydrate was prepared as follows: Amorphous Compound 1 (1.0 g) and ethyl acetate (25 mL) were added to a 50 mL reactor vessel. Compound 1 dissolved under 25° C., and the agitation rate was kept at 300 rpm (two-blade paddle). Fumaric acid (1.2 equiv) was added in one portion. After stirring for 10 min, a large amount of solid precipitated. The mixture was stirred for 15 h. The suspension was filtered, and the wet cake dried at 40° C. for 16 h in a vacuum oven to give Compound 1 Fumarate Form A Anhydrate (1.035 g, 82% yield).

The results are summarized in Table 1. Amorphous Compound 1 had high solubility in isopropanol, and fumaric acid had relatively low solubility in isopropanol. Compound 1 Fumarate Form E had very low solubility in isopropanol at 25° C., and the solubility decreased significantly with increasing proportion of heptane. Compound 1 Fumarate Form E had much lower solubility than Compound 1 Fumarate Form A Anhydrate, indicating that Form E is more stable than Form A.

TABLE 1

| Material | Method | Solvent | Solubility (mg/mL) | |
|---|---|---|---|---|
| Amorphous | Dynamic | IPA | >467.5 | |
| Compound 1 | | | | |
| Fumaric acid | Dynamic | IPA | 34.3 | |
| Compound 1 | Dynamic | IPA | ~8.46 | (25° C.) |
| Fumarate Form | Dynamic | IPA | <1 | (0° C.) |
| E | Dynamic | IPA/heptane (5/1) | 5.89 | |
| | Dynamic | IPA/heptane (5/3) | ~3.57 | |
| | Dynamic | IPA/heptane (1/1) | ~2.35 | |
| | Dynamic | IPA/heptane (1/3) | <1 | |
| | Gravimetric | H₂O | 1.0 | |
| Compound 1 | Gravimetric | H₂O | 6.5 | |
| Fumarate Form | | | | |
| A Anhydrate | | | | |

### Example 4: Bulk Density Studies

Bulk density of Compound 1 Fumarate Form E was measured by gently introducing a known sample mass into a graduated cylinder (50 mL), leveling the powder without compacting it, and recording the apparent untapped volume to the nearest graduated unit. The experiment was repeated three times, and the results are summarized in Table 2.

TABLE 2

| Run | Bulk Density (g/mL) |
|---|---|
| 1 | 0.1288 |
| 2 | 0.1233 |
| 3 | 0.1196 |
| Average | 0.1239 |

### Example 5: Stability Studies

Amorphous Compound 1, Compound 1 Fumarate Form E, and Compound 1 Fumarate Form A Anhydrate were placed in an oven at 60° C. for two weeks to evaluate their stability. As shown in Table 3, Compound 1 Fumarate Form E and Compound 1 Fumarate Form A Anhydrate exhibited improved stability relative to Amorphous Compound 1.

TABLE 3

| | HPLC Purity (%) | | | |
|---|---|---|---|---|
| Material | Starting | 60° C. | Degradation | XRPD |
| Amorphous | 99.23% | 98.94% | −0.29% | No change |
| Compound 1 | | | | |
| Compound 1 | 99.54% | 99.50% | −0.05% | No change |
| Fumarate Form E | | | | |
| Compound 1 | 99.58% | 99.54% | −0.04% | No change |
| Fumarate Form A | | | | |
| Anhydrate | | | | |

Competitive slurry experiments were performed with Compound 1 Fumarate Form E and Compound 1 Fumarate Form A Anhydrate, as follows:

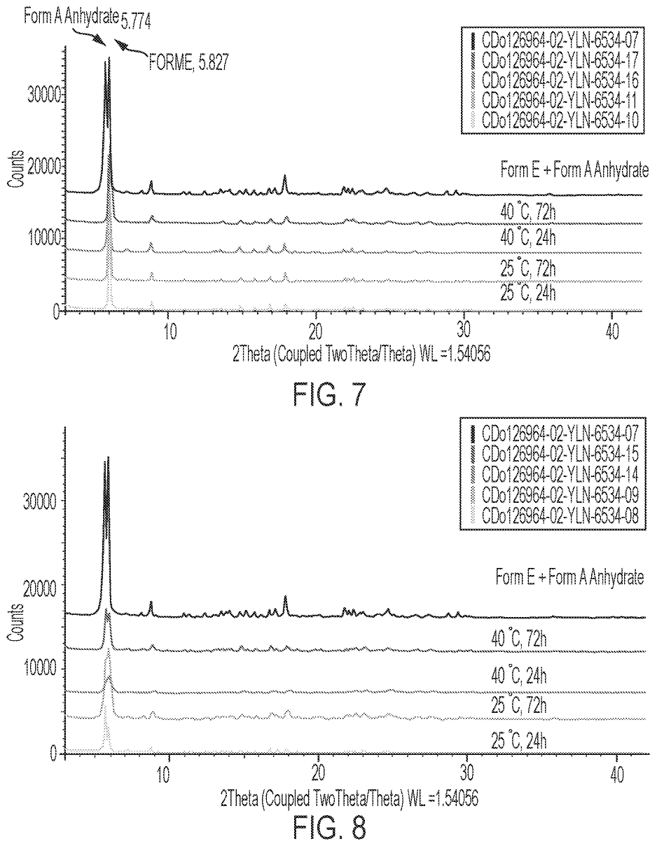

Experiment #1: Compound 1 Fumarate Form E (100 mg), Compound 1 Fumarate Form A Anhydrate (100 mg), and isopropanol (2 mL) were added into two 8-mL vials and mixed well in a shaker at 25° C. and 40° C., respectively. Samples for analysis were taken at 24 h and 72 h. Results of XRPD analysis are shown in FIG. 7. After 24 h at either 25° C. or 40° C., Compound 1 Fumarate Form A Anhydrate completely transformed into Compound 1 Fumarate Form E, indicating that Form E is more stable than Form A Anhydrate in isopropanol.

Experiment #2: Compound 1 Fumarate Form E (100 mg), Compound 1 Fumarate Form A Anhydrate (100 mg), and water (2 mL) were added into two 8-mL vials and mixed well in a shaker at 25° C. and 40° C., respectively. Samples for analysis were taken at 24 h and 72 h. Results of XRPD analysis are shown in FIG. 8. After 72 h at either 25° C. or 40° C., a mixture of Compound 1 Fumarate Form E and Compound 1 Fumarate Form A Anhydrate remained, indicating that conversion between the forms is very slow in water.

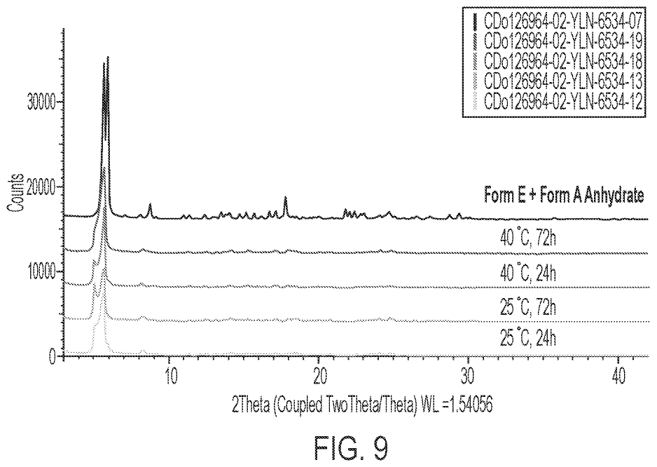

Experiment #3: Compound 1 Fumarate Form E (100 mg), Compound 1 Fumarate Form A Anhydrate (100 mg), and ethyl acetate (2 mL) were added into two 8-mL vials and mixed well in a shaker at 25° C. and 40° C., respectively. Samples for analysis were taken at 24 h and 72 h. Results of XRPD analysis are shown in FIG. 9. After 72 h at either 25° C. or 40° C., a mixture of Compound 1 Fumarate Form E and Compound 1 Fumarate Form A Anhydrate remained, indicating that conversion between the forms is slow in ethyl acetate.

### Example 6: Polymorph Screening of Compound 1

Polymorph screening of Compound 1 was performed under 100 experimental conditions starting with amorphous Compound 1. A total of eight screening methods were used, including anti-solvent addition, reverse anti-solvent addition, slurry at 5° C., slurry at RT, slow evaporation, slow cooling, temperature cycling, and solid vapor diffusion. Polymorph screening identified at least two crystalline forms of Compound 1, both of which were solvates. Form A was found to exist as multiple different isostructural solvates (e.g., acetonitrile, acetone, and tetrahydrofuran solvates). Form B was determined to be a DMSO solvate. The results are summarized in Table 4, below:

TABLE 4

| | No. of | |
|---|---|---|
| Method | Experiments | Crystal Form(s) Identified |
| Anti-solvent addition | 8 | Amorphous |
| Slow evaporation | 9 | Form A and Amorphous |
| Slow cooling | 11 | Form A and Amorphous |
| Slurry at RT | 30 | Form A, Form B, and Amorphous |
| Slurry at 5° C. | 11 | Form A and Amorphous |
| Solid vapor diffusion | 15 | Form A, Form B, and Amorphous |
| Reverse anti-solvent | 8 | Amorphous |
| addition | | |
| Temperature cycling | 8 | Form A and Amorphous |
| Total | 100 | |

Anti-solvent addition experiments were conducted under 8 conditions, respectively. About 15 mg of Compound 1 was dissolved in 0.4-3.0 mL solvent to obtain a clear solution. The solution was magnetically stirred followed by addition of 0.1 mL anti-solvent per step for first 1 mL and adding 0.5 mL stepwise until precipitate appeared, or the total amount of anti-solvent reached 5.0 mL. The obtained precipitate was isolated for XRPD analysis. As summarized in Table 5, only amorphous Compound 1 was observed.

TABLE 5

| Solvent | Anti-Solvent | Form Observed |
|---|---|---|
| MeOH | H₂O | Amorphous |
| EtOH | H₂O | Amorphous |
| IPA | H₂O | Amorphous |
| Acetone | H₂O | Amorphous |
| THF | H₂O | Amorphous |
| ACN | H₂O | Amorphous |
| DMSO | H₂O | Amorphous |
| DMF | H₂O | Amorphous |

Slow evaporation experiments were conducted at RT under 9 different conditions. About 15 mg of Compound 1 was dissolved in 0.5 mL of solvent. All solutions and suspensions were filtered using a 0.45 μm PTFE membrane the filtrates were used for the following steps. The visually clear solutions were covered by a HPLC cap with a hole in the cap created by a pipette tip and subjected to evaporation at room temperature. The solids were isolated for XRPD analysis. The result, summarized in Table 6, showed that Form A was obtained under certain conditions:

TABLE 6

| Solvent (v/v) | Form Observed |
|---|---|
| ACN | Form A |
| MeOH | Amorphous |
| EtOH | Amorphous |
| Acetone | Amorphous |
| THF | Gel |
| IPA | Gel |
| EtOAc | Gel |
| CHCl₃ | Gel |
| DCM | Gel |

Slow cooling experiments were conducted in 11 solvent systems, respectively. About 20 mg of Compound 1 was dissolved in 1.0-2.0 mL of solvent at 60° C. and filtered to a new vial using a 0.45 μm PTFE membrane. Filtrates were slowly cooled down from 60° C. to 5° C. at a rate of 0.05° C./min. The obtained solids were kept isothermal at 5° C. before isolated for XRPD analysis. Anti-solvents were added into clear solutions to induce precipitation. Slow evaporation was conducted if no solid was observed after addition of anti-solvent. Results, summarized in Table 7, showed that Form A was obtained under certain conditions:

TABLE 7

| Solvent (v/v) | Form(s) Observed |
|---|---|
| ACN | Form A |
| MeOH/H₂O (1:4) | Amorphous* |
| EtOH/H₂O (1:4) | Amorphous* |
| Acetone/H₂O (1:4) | Form A + Amorphous* |
| ACN/H₂O (1:4) | Form A |
| THF/H₂O (1:4) | Form A + Amorphous* |
| MeOH/H₂O (1:9) | Amorphous* |
| EtOH/H₂O (1:9) | Amorphous* |
| Acetone/H₂O (1:9) | Amorphous* |
| ACN/H₂O (1:9) | Form A |
| THF/H₂O (1:9) | Amorphous* |

*Slow evaporation procedures used, as described above.

Slurry conversion experiments were conducted at RT in 30 solvent systems. About 20 mg of Compound 1 was suspended in 0.3 mL of solvent at RT for 4 days. The remaining solids were isolated for XRPD analysis. Results, summarized in Table 8, indicated that Form A and Form B were obtained under certain conditions:

TABLE 8

| Solvent (v/v) | Form Observed |
|---|---|
| ACN | Form A |
| DMSO | Form B |
| H₂O | Amorphous |
| DMSO/H₂O (1:1) | Amorphous |
| EtOH/H₂O (1:1) | Amorphous |
| Acetone/H₂O (1:1) | Form A |
| ACN/H₂O (1:1) | Form A |
| THF/H₂O (1:1) | Amorphous |
| DMSO/H₂O (1:4) | Amorphous |
| EtOH/H₂O (1:4) | Amorphous |
| Acetone/H₂O (1:4) | Amorphous |
| ACN/H₂O (1:4) | Form A |
| THF/H₂O (1:4) | Form A |
| DMSO/H₂O (1:9) | Amorphous |
| EtOH/H₂O (1:9) | Amorphous |
| Acetone/H₂O (1:9) | Amorphous |
| ACN/H₂O (1:9) | Form A |
| THF/H₂O (1:9) | Form A |
| ACN/H₂O (aw = 0.2, 989/11)* | Form A |
| ACN/H₂O (aw = 0.4, 978/22)* | Form A |
| ACN/H₂O (aw = 0.6, 959/41)* | Form A |
| ACN/H₂O (aw = 0.8, 925/75)* | Form A |
| Acetone/H₂O (aw = 0.2, 941/59)* | Form A |
| Acetone/H₂O (aw = 0.4, 857/143)* | Form A |
| Acetone/H₂O (aw = 0.6, 726/274)* | Form A |
| Acetone/H₂O (aw = 0.8, 492/508)* | Form A |
| DMSO/H₂O (aw = 0.2, 842/158) | Amorphous |
| DMSO/H₂O (aw = 0.4, 710/290) | Amorphous |
| DMSO/H₂O (aw = 0.6, 570/430) | Amorphous |
| DMSO/H₂O (aw = 0.8, 373/627) | Amorphous |

*Theoretical water activity based on software simulation.

Slurry conversion experiments were conducted at 5° C. in 11 solvent systems. About 30 mg of Compound 1 was suspended in 0.3 mL of solvent at 5° C. for 4 days. The remaining solids were isolated for XRPD analysis. Results, summarized in Table 9, indicated that Form A was obtained under certain conditions:

TABLE 9

| Solvent (v/v) | Form Observed |
|---|---|
| ACN | Form A |
| DMSO | Low Crystallinity* |
| ACN/H₂O (aw = 0.2, 989/11) | Form A |
| ACN/H₂O (aw = 0.4, 978/22) | Form A |
| ACN/H₂O (aw = 0.6, 959/41) | Form A |
| ACN/H₂O (aw = 0.8, 925/75) | Form A |
| DMSO/H₂O (1:4) | Amorphous |
| EtOH/H₂O (1:4) | Amorphous |
| Acetone/H₂O (1:4) | Form A |
| ACN/H₂O (1:4) | Form A |
| THF/H₂O (1:4) | Form A |

*Slow evaporation procedures used.

Solid vapor diffusion experiments were conducted using 12 different solvents, respectively. About 15 mg of Compound 1 was weighed into a 3-mL vial, which was placed into a 20-mL vial with 4 mL of volatile solvent. The 20-mL vial was sealed with a cap and kept at RT for 9 days allowing solvent vapor to interact with sample. The solids were tested by XRPD. The results, summarized in Table 10, showed that Form A and Form B were obtained under certain conditions:

TABLE 10

| Solvent | Form Observed |
|---|---|
| H₂O | Amorphous |
| MeOH | Amorphous |
| EtOH | Gel |
| IPA | Gel |
| Acetone | Gel |
| MEK | Gel |
| MIBK | Gel |
| IPAc | Gel |
| THF | Gel |
| 2-MeTHF | Gel |
| MTBE | Gel |
| DCM | Gel |
| ACN | Form A |
| Toluene | Gel |
| DMSO | Form B |

Reverse anti-solvent addition experiments were conducted in 8 solvent systems by first placing 1 mL of anti-solvent into a refrigerator at 5° C. in a 3 mL glass vial. About ~10 mg of Compound 1 was then dissolved in 1 mL of solvent in a 2-mL glass vial. After the suspension was stirred magnetically for 2 hours yielding a clear solution, the solution was quickly filtered into the 5° C. antisolvent. The sample was then left at 5° C. to crystallize. If no crystallization occurred after 1 day, the sample was moved to −20° C. to precipitate. Remaining solids were isolated for XRPD analysis. The results, summarized in Table 11, showed that only amorphous Compound 1 was obtained:

TABLE 11

| Solvent | Anti-Solvent | Form Observed |
|---|---|---|
| MeOH | H₂O | Amorphous |
| EtOH | H₂O | Amorphous |
| IPA | H₂O | Amorphous |
| Acetone | H₂O | Amorphous |
| THF | H₂O | Amorphous |
| ACN | H₂O | Amorphous |
| DMSO | H₂O | Amorphous |
| DMF | H₂O | Amorphous |

Temperature cycling experiments were conducted in 8 solvent systems. About 20 mg of Compound 1 was suspended in 0.1 mL of solvent in a 23-mL glass vial at RT. The suspension was then heated to 60° C., equilibrated for two hours. The slurry was slowly cooled down to 5° C. at a rate of 0.1° C./min and then heat to 60° C. in one hour. Repeat the cycle one more time and then cooling to 5° C. at a rate of 0.1° C./min. The samples were stored 5° C. before solids were isolated and analyzed using XRPD. Results summarized in Table 12 showed that Form A was obtained.

TABLE 12

| Solvent (v/v) | Form Observed |
|---|---|
| ACN | Form A |
| H₂O | Amorphous |
| EtOH/H₂O (1:4) | Amorphous |
| DMSO/H₂O (1:4) | Amorphous |
| ACN/H₂O (aw = 0.2, 989/11) | Form A |
| ACN/H₂O (aw = 0.4, 978/22) | Form A |
| ACN/H₂O (aw = 0.6, 959/41) | Form A |
| ACN/H₂O (aw = 0.8, 925/75) | Form A |

### Example 7: Salt Screening of Compound 1

Salt screening was conducted at room temperature (RT). A total of 100 salt screening experiments were conducted using 25 acids in 4 different solvent systems. Specifically, the stock solutions of Compound 1 are summarized in Table 13. The summary of the salt screen is presented in Table 14.

TABLE 13

| | | | | Volume used |
|---|---|---|---|---|
| | | Actual | Total | for each |
| | Temperature | sample size | volume | screening exp |
| Solvent (v/v) | (° C.) | (mg) | (mL) | (mL) |
| EtOH | RT | 610.1 | 15 | 0.5 |
| ACN | RT | 605.6 | 15 | 0.5 |
| EtOAc | RT | 611.7 | 15 | 0.5 |
| Acetone/H₂O | RT | 602.3 | 15 | 0.5 |
| (19:1, v:v) | | | | |

The stock solutions of Compound 1 were prepared in solvent systems at RT.
Sonication and heating were applied to the stock solutions until all Compound 1 was dissolved.

TABLE 14

| | Solvent | | | | |
|---|---|---|---|---|---|
| | | | | | Acetone/H₂O |
| Co-Former | pKa | EtOH | ACN | EtOAc | (19:1, v:v) |
| Blank | NA | Amorphous | Free Base Form A | Amorphous | Amorphous |
| (−)-L-Maleic acid (2:1) | 3.46 | Gel | Gel | Gel | Gel |
| (+)-D-Maleic acid (2:1) | 3.46 | Malate Form A | Gel | Malate Form A | Gel |
| (+)-L-Tartaric acid (2:1) | 3.02 | Gel | Gel | Gel | Gel |
| (−)-D-Tartaric acid (2:1) | 3.02 | Gel | Gel | Gel | Gel |
| Citric acid (2:1) | 3.13 | Gel | Gel | Citric Acid | Gel |
| D-Glucuronic Acid (2:1) | 3.18 | D-Glucuronic Acid | D-Glucuronic Acid | D-Glucuronic Acid | D-Glucuronic Acid |
| Fumaric acid (2:1) | 3.03 | Gel | Fumarate Form A | Fumarate Form A | Fumarate Form A |
| Glutamic acid (2:1) | 2.19 | Glutamic acid | Glutamic acid | Glutamic acid | Glutamic acid |
| Glycolic Acid (2:1) | 3.28 | Gel | Gel | Gel | Gel |
| H₃PO₄ (1:1) | 1.96 | Phosphate Form B | Gel | Phosphate Form A | Gel |
| H₃PO₄ (2:1) | 1.96 | Gel | Gel | Gel | Gel |
| HCl (1:1) | −6.00 | Gel | Gel | Gel | Gel |
| HCl (2:1) | −6.00 | Gel | Gel | Gel | Gel |
| Hippuric acid (2:1) | 3.55 | Gel | Hippuric acid | Hippuric acid | Gel |
| L-Aspartic acid (2:1) | 1.88 | L-Aspartic acid | L-Aspartic acid | L-Aspartic acid | L-Aspartic acid |
| Maleic acid (2:1) | 1.92 | Gel | Gel | Gel | Gel |
| Sulfuric acid (1:1) | −3.00 | Gel/Oil* | Oil* | Oil* | Gel/Oil* |
| Sulfuric acid (2:1) | −3.00 | Gel/Oil* | Oil* | Oil* | Gel/Oil* |
| (−)-L-Pyroglutamic acid | 3.32 | Gel | Gel | Gel | Gel |
| (2:1) | | | | | |
| Benzenesulfonic Acid | 0.70 | Gel | Gel | Gel | Gel |
| (2:1) | | | | | |
| Fumaric acid (1:1) | 3.03 | Gel | Fumarate Form A | Fumarate Form A | Gel |
| Malonic Acid (2:1) | 2.83 | Gel | Gel | Gel | Gel |
| Methanesulfonic acid | −1.20 | Gel | Gel | Gel | Gel |
| (2:1) | | | | | |
| Oxalic Acid (2:1) | 1.27 | Gel | Gel | Oxalate Form A | Oxalate Form A |
| Pamoic Acid (2:1) | 2.51 | Pamoic Acid | Pamoic Acid | Pamoic Acid | Pamoic Acid |

*Sample slurried for 1 hour at 5° C. and became black gel/oil.

All 14 hits were characterized by XRPD, TGA, DSC and solution NMR. The characterization results are summarized in Table 15.

TABLE 15

| | | | | Stoichiometry |
|---|---|---|---|---|
| | | | Purity | by NMR |
| | Weight Loss | DSC Endo. | by | (Counter Ion |
| Form | in TGA (%) | (Peak, ° C.) | HPLC | to Compound 1) |
| Malate | 1.58% | 60.7° C. and | ~100% | 2:1 |
| Form A | | 157.1° C. (peak) | | |
| Fumarate | 0.2% | 139.5° C. | ~100% | 1:1 |
| Form A | | | | |
| Anhydrate | | | | |
| Phosphate | 2.47% | 62.9° C. (peak), | 99.72% | ND |
| Form A | | 168.6° C. (onset) | | |
| | | and | | |
| | | 186.6° C. (peak). | | |
| Phosphate | 3.98% | 38.3° C., | 99.36% | ND |
| Form B | | 132.9° C. and | | |
| | | 156.1° C. (peak). | | |
| Oxalate | 0.97% | 94.0° C. (peak) | 99.77% | ND |
| Form A | | and | | |
| | | 125.7° C. (onset). | | |

ND = not determined.

Three salts were selected for further characterization—a malate salt, a fumarate salt, and an oxalate salt. The salts were scaled up to hundreds of milligrams. Characterization data are summarized in Table 16.

TABLE 16

| | Amorphous | | Fumarate Form A | |
|---|---|---|---|---|
| Crystal Form | Freebase | Malate Form A | Anhydrate | Oxalate Form A |
| Crystallinity | Amorphous | High | High | Medium |
| Weight Loss in TGA | 0.38% | 1.58% | 0.2% | 0.97% |
| (%) | | | | |
| Endotherm in DSC | 73.3 | 60.7, 157.1 | 139.5 | 94.0, 125.7 |
| (° C., peak) | | | | |
| HPLC purity (area %) | 100.00 | 100.00 | 100.00 | 99.77 |
| Stoichiometry | N/A | 2.06 | 0.965 | N/A |
| (acid:Compound 1) | | | | |
| Hygroscopicity (%)* | Slightly | Hygroscopic (8.38) | Non- | Hygroscopic (10.62) |
| | hygroscopic (0.463) | | hygroscopic (0.108) | |
| Form Change after | No | No | No | No |
| DVS? | | | | |

*Based on water uptake up at 25° C./80% RH: very hygroscopic: >15%, hygroscopic: 2-15%, slightly hygroscopic: 0.2-2%, non-hygroscopic: <0.2%.

### Example 8: Additional Polymorph Screening of Compound 1

A polymorph screen was conducted using amorphous Compound 1. To help design the experiments, kinetic solubilities of the compound were estimated. The estimation was done using a solvent aliquot addition method, and dissolution was judged by visual observation. Results are provided in Table 17. In Table 17, solvent ratios (v/v) are approximate; values are rounded to nearest whole number. If complete dissolution was achieved by one aliquot addition, solubilities were reported as “>”; if no solids were present, solubilities were reported as “<”. The actual solubility may be larger than the value calculated due to the use of solvent aliquots that were too large or due to a slow rate of dissolution.

TABLE 17

| Solvent System | Solubility (mg/mL) |
|---|---|
| ACN/water (60/40) | <6 |
| Dioxane/water (60/40) | <13 |
| Heptane | >191 |
| IPA | 82 |
| MeOH/water (40/60) | <55* |
| 2-MeTHF/MeOH/water (60/20/20) | 106 |
| NMP/water (68/32) | 7 |

*Appeared hydrophobic

Based on the solubility data, crystallization experiments were designed at micro (~5-10 mg) and medium (~30-80 mg) scales, utilizing techniques such as slow evaporation, slurry, and vapor stress of melts. Addition of crystalline seeds and a selected salt former were also explored. The experiments consisted of multiple steps, where observations from initial steps guided the approach to the subsequent steps.

Samples generated were visually observed by polarized light microscopy and analyzed by XRPD to perform a preliminary assessment. If solids produced exhibited a unique XRPD pattern, they were further characterized by solution 1H NMR to confirm the chemical composition and by TGA and DSC to evaluate the thermal behavior and the presence of volatiles. Conditions and results of the screen are summarized in Table 18.

TABLE 18

| Solvent System (Scale) | Conditions | Observations | XRPD Results |
|---|---|---|---|
| ACN | 1) Supernatant from | 1) Clear | Amount insufficient |
| | synthesis of Compound 1 | 2) Crystals | for XRPD |
| | Form A Acetonitrile Solvate | | |
| | 2) Slow evaporation | | |
| ACN/water (60/40) | 1) Added solvent | 1) Partially dissolved, turned | Amount insufficient |
| (microscale) | 2) Kept at RT, 9 days | viscous | for XRPD |
| | | 2) Crystalline, unknown | |
| | | morphology, BE | |
| BuOH dry | 1) Heated until liquefied, | 1) Crystalline, prismatic, BE | Amount insufficient |
| (medium scale) | cooled to RT, vapor stress | | for XRPD |
| | 1) Added solvent | 1) Dissolved | — |
| | 2) Slow evaporation | 2) Film | |
| DCE | 1) Added solvent | 1) Dissolved, no BE | — |
| (medium scale) | 2) Slow evaporation | 2) Film, no BE | |
| | 3) Scuffed bottom, kept at | 3) No change | |
| | RT | | |
| Dioxane/water (60/40) | 1) Added solvent | 1) Partially dissolved, turned | Amount insufficient |
| (microscale) | 2) Kept at RT, 9 days | viscous | for XRPD |
| | | 2) Crystalline, unknown | |
| | | morphology, BE | |
| Dioxane/water (60/40) | 1) Added solvent | 1) Viscous | Crystalline, similar to |
| (medium scale) | 2) Slurry, RT, 6 days | 2) Unknown morphology, BE | Compound 1 Form A |
| | | not apparent | Acetonitrile Solvate |
| | | | (Sample 15.1) |
| EtOAc dry | 1) Added solvent | 1) Dissolved, no BE | — |
| (medium scale) | 2) Slow evaporation | 2) Film, no BE | |
| | 3) Scuffed bottom, kept at | 3) Film | |
| | RT | | |
| | 1) Heated until liquefied, | 1) Potential crystalline, solution, | Crystalline, similar to |
| | cooled to RT, vapor stress | few BE speces | Compound 1 Form A |
| | 2) Slow evaporation | 2) Crystalline, BE, potential for | Acetonitrile Solvate |
| | 3) Attempted to sample | singles | (Sample 15.2) |
| | crystals for SCXRD | 3) Crystals broke | |
| EtOAc/heptane dry | 1) Added solvent | 1) Dissolved | — |
| (medium scale) | 2) Slow evaporation | 2) Film | |
| Heptane dry | 1) Heated until liquefied, | 1) — | — |
| (medium scale) | cooled to RT | 2) Dissolved, no BE | |
| | 2) Triturated with solvent | 3) Film, no BE | |
| | 3) Slow evaporation | 4) Film | |
| | 4) Scuffed bottom, kept at | | |
| | RT | | |
| | 1) Heated until liquefied, | 1) — | — |
| | cooled to RT | 2) Dissolved, no BE | |
| | 2) Vapor stress | 3) No solids | |
| | 3) Slow evaporation | | |
| | 1) Added solvent | 1) Dissolved, no BE | — |
| | 2) Fast evaporation, | 2) Film, no BE | |
| | elevated temperature | | |
| Isopropyl ether | 1) Added solvent | 1) Mostly clear, some viscous | — |
| | 2) Added crystal from Row | 2) Crystal remained | |
| | 1 (ACN experiment) | 3) No further change | |
| | 3) Kept at RT | | |
| IPA | 1) Added solvent | 1) Dissolved, no BE | — |
| (microscale) | 2) Kept at RT, 9 days | 2) Evaporated, film, no BE | |
| | 3) Scuffed bottom, kept at | 3) No change | |
| | RT | | |
| IPA | 1) Added solvent | 1) Dissolved, no BE | — |
| (medium scale) | 2) Slow evaporation | 2) Film | |
| | 1) Heated until liquefied, | 1) — | — |
| | cooled to RT | 2) Dissolved, no BE | |
| | 2) Vapor stress | 3) Film | |
| | 3) Slow evaporation | | |
| IPA/water (50/50) | 1) Added solvent | 1) Partially dissolved, turned | — |
| (medium scale) | 2) Kept at RT, 9 days | viscous | |
| | 3) Kept at RT | 2) Viscous, no BE | |
| | | 3) No change | |
| MeOH | 1) Added solvent | 1) Dissolved | — |
| (medium scale) | 2) Slow evaporation | 2) Viscous, no BE | |
| MeOH/water (40/60) | 1) Added solvent | 1) Appeared hydrophobic | — |
| (medium scale) | 2) Slurry, RT | 2) No BE | |
| 2-MeTH | 1) Heated until liquefied, | 1) — | — |
| (medium scale) | cooled to RT | 2) Dissolved, no BE | |
| | 2) Vapor stress | 3) Film, no BE | |
| | 3) Slow evaporation | | |
| | 1) Added solvent | 1) Dissolved, no BE | — |
| | 2) Slow evaporation | 2) Potential crystalline, film, few | |
| | 3) Scuffed bottom, kept at | BE particles | |
| | RT | 3) No change | |
| 2-MeTHF/MeOH/water | 1) Added solvent | 1) Dissolved, no BE | — |
| (60/20/20) | 2) Kept at RT, 9 days | 2) Partially evaporated, oil, no | |
| (microscale) | | BE | |
| MIBK dry | 1) Heated until liquefied, | 1) — | — |
| (medium scale) | cooled to RT | 2) Dissolved, no BE | |
| | 2) Vapor stress | 3) No solids | |
| | 3) Slow evaporation | | |
| MTBE | 1) Heated until liquefied, | 1) — | — |
| (medium scale) | cooled to RT | 2) Dissolved, no BE | |
| | 2) Vapor stress | 3) Film | |
| | 3) Slow evaporation | | |
| | 1) Added solvent | 1) Dissolved, no BE | — |
| | 2) Slow evaporation | 2) Potential crystalline, film, few | |
| | 3) Scuffed bottom, kept at | BE particles | |
| | RT | 3) No change | |
| NMP/water (68/32) | 1) Added solvent | 1) Dissolved, no BE | — |
| (microscale) | 2) Kept at RT, 9 days | 2) Partially evaporated, | |
| | 3) Sonicated, placed in | crystalline, unknown | |
| | freezer | morphology, BE | |
| | | 3) No change | |
| NMP/water (60/40) | 1) Added solvent | 1) Viscous | Crystalline, similar to |
| (medium scale) | 2) Slurry, RT, 6 days | 2) Crystalline, unknown | Compound 1 Form A |
| | | morphology, BE | Acetonitrile Solvate |
| | | | (Sample 15.3) |
| Water | 1) Heated until liquefied, | 1) — | — |
| (medium scale) | cooled to RT | 2) Glassy, no BE | |
| | 2) Vapor stress | 3) No change | |
| | 3) Kept at RT | | |
| Salt former addition | 1) Mixed with L-tartaric | 1) Remained | — |
| | acid | 2) Dissolved | |
| | 2) Added MeOH | 3) Evaporated, yellow viscous | |
| | 3) Slow evaporation | substance | |
| | 4) Added MTBE | 4) No change | |
| | 5) Added heptane | 5) Some solids | |
| | (MTBE/heptane, 29/71) | 6) Oil | |
| | 6) Slurry, RT, 11 days | 7) No change | |
| | 7) Decanted, added | | |
| | NMP/water, slurry | | |

BE = birefringence/extinction;
SCXRD = single crystal X-ray diffraction

Table 19 provides a summary of characterization data for the materials produced from this experiment. Sample numbers reference Table 15.

TABLE 19

| Sample | Analytical Technique | Results |
|---|---|---|
| Sample 15.2 | XRPD | Crystalline; single crystalline phase based on XRPD pattern |
| | | indexing; unit cell volume consistent with one molecule of |
| | | EtOAc per Compound 1 molecule. |
| | | Small peak at 5.4° 2θ due to Kapton. |
| Sample 15.1 | XRPD | Crystalline; single crystalline phase based on XRPD pattern |
| | | indexing; unit cell volume consistent with one molecule of |
| | | dioxane per Compound 1 molecule. |
| | | Small peak at 5.4° 2θ due to Kapton. |
| | ¹H NMR (collected 5 days | Consistent with chemical structure of Compound 1. |
| | after XRPD) | Dioxane (0.5 mol/mol) based on peak at 3.57 ppm. |
| | TGA (collected 5 days | Continuous loss of volatiles begins at 72° C.: 8.7 wt % loss, 72- |
| | after XRPD) | 221° C., equivalent to 0.5 mol dioxane. |
| | | Weight loss, onset at 252° C. |
| | DSC (collected 5 days after | Endotherm, peak max at 126° C. |
| | XRPD) | Exotherm, above 200° C. |
| Sample 15.3 | XRPD | Crystalline; similar to Compound 1 Form A Acetonitrile |
| | | Solvate. |
| | | Small peak at 5.4° 2θ due to Kapton. |
| | ¹H NMR (collected 4 days | Consistent with chemical structure of Compound 1. |
| | after XRPD) | NMP (0.5 mol/mol) based on peaks at 2.69, 2.18, and 1.91 ppm |
| | TGA (collected 5 days | Continuous loss of volatiles begins at 89° C.: 9.0 wt % loss, 89- |
| | after XRPD) | 239° C., equivalent to 0.5 mol NMP. |
| | | Weight loss, onset at 274° C. |
| | DSC (collected 5 days after | Endotherm, peak max at 95° C. |
| | XRPD) | Exotherm, above 200° C. |

### Example 9: Polymorph Screening of Compound 1 Fumarate

A polymorph screen was conducted using Compound 1 Fumarate Form A Ethyl Acetate Solvate. Form A Ethyl Acetate Solvate was prepared as follows: Amorphous Compound 1 (3.0025 g) was suspended in ethyl acetate (60 mL) resulting in a clear solution. Fumaric acid (774.6 mg) was added to the solution, an additional precipitation was observed. The mixture was stirred at ambient temperature for approximately a week. The solids formed were isolated by filtration via syringe with a positive displacement. Approximately 4.5 g of undried solids were recovered.

The screen consisted primarily of long term slurry experiments. To help design screen experiments, kinetic solubilities of Fumarate Form A Ethyl Acetate Solvate were estimated. The estimation was done on a 3-11 mg scale using a solvent aliquot addition method, and dissolution was judged by visual observation. Results are provided in Table 20. Solubilities are estimated at ambient temperature and reported to the nearest mg/mL; if complete dissolution was achieved by one aliquot addition, solubilities were reported as “>”;

TABLE 20

| Solvent System (Ratio v %) | Solubility (mg/mL) |
|---|---|
| ACN | <2* |
| Anisole | <1 |
| t-BuOAc | 1* |
| 2-BuOH | 17 |
| DCE | <9 |
| DCE/EtOH (96/4) | Dissolved at 8 |
| Dioxane | >104 |
| IPA | 20 |
| IPA/water (50/50) | >124 |
| 2-MeTHF | 32 |
| MIBK | 4 |
| MTBE | 2 |
| TFE | >100 |

*Re-precipitated after first aliquot.

Long-term slurry experiments were conducted by stirring suspensions of Compound 1 Fumarate Form A Ethyl Acetate Solvate (~30-100 mg) in various solvent systems at ambient temperaturc. Solvent systems were selected based on solubility estimations. After 20 days of stirring, solids were isolated by centrifugation with filtration (Table 21).

TABLE 21

| Solvent System | | | |
|---|---|---|---|
| (Ratio v %) | Conditions | Observations | XRPD Results |
| ACN | RT, 20 days | White solids | Fumarate Form G |
| tBuOAc | RT, 20 days | White solids | Fumarate Form F + |
| | | | Kapton peak at 5° 2θ |
| 2-BuOH | RT, 20 days | Light yellow | Fumarate Form E + |
| | | solids | Kapton peak at 5° 2θ |
| EtOH/DCE | RT, 20 days | White solids | Fumarate Form H + |
| (4/96) | | | Fumarate Form E |
| IPA | RT, 20 days | White solids | Fumarate Form E + |
| | | | Kapton peak at 5° 2θ |
| MEK/heptane | RT, 20 days | White solids | Fumarate Form H + |
| (40/60) | | | Kapton peak at 5° 2θ |
| 2-MeTHF | RT, 20 days | White solids | Fumarate Form H |
| MIBK | RT, 20 days | Light yellow | Fumarate Form D |
| | | solids | |
| MTBE | RT, 20 days | White solids | Fumarate Form H |
| NMP/water | RT, 20 days | White solids | Fumarate Form E + |
| (40/60) | | | Kapton peak at 5° 2θ |

Kinetic experiments using ~30-50 mg Compound 1 Fumarate Form A Ethyl Acetate Solvate included crystallization techniques such as fast and slow evaporation; solvent/antisolvent precipitation with ripening; crystallization at subambient temperature; and organic and aqueous vapor stress (Table 22). For evaporation experiments, filtered solutions of test material were left uncapped at ambient temperature for fast evaporation or covered with aluminum foil with pin holes for slow evaporation. For solvent/antisolvent precipitation, solutions of starting material were prepared at ambient or elevated temperature and filtered using a 0.2 μm nylon filter. The solutions were mixed with appropriate antisolvents via a direct or reverse addition. Solids precipitated were either immediately isolated by vacuum filtration or left at ambient temperature for ripening. For crystallization at subambient temperature, solutions of starting material were prepared at ambient temperature and filtered using a 0.2 μm nylon filter. The filtered solutions were then placed at sub-ambient conditions for slow crystallization. Solids precipitated were isolated via centrifugation with filtration. For vapor stress experiments, solids of starting material were sampled in vials, which were placed in a RH jar (prepared as described in Greenspan, L., Journal of Research of the National Bureau of Standards Section A: Physics and Chemistry, vol. 81A, no. 1, 1977, p. 89, doi:10.6028/jres.081a.011) at set temperature or a secondary container with water. After a specified duration, samples were collected and analyzed.

TABLE 22

| Solvent System | | | |
|---|---|---|---|
| (Ratio v %) | Conditions | Observations | XRPD Results |
| 2-BuOH | Crystallization in freezer in | Clear | — |
| | attempt to grow suitable | | |
| | crystals (6 mg scale) | | |
| Dioxane/hexanes | Solvent/antisolvent | Unknown | Fumarate Form H |
| (8/92) | precipitation with ripening | morphology, sticky, | |
| | | some BE | |
| DCE/EtOH (96/4) | Slow evaporation in attempt to | Crystals with BE; not | — |
| | grow suitable crystals (11 mg | suitable for SCXRD | |
| | scale) | | |
| EtOH/nitromethane | Slow evaporation | Film, no BE | — |
| (3/97) | | | |
| EtOH/toluene (9/91) | Slow evaporation | Unknown | Fumarate Form B + Kapton |
| | | morphology, BE | peaks at 5, 15, and 29° 2θ |
| IPA/heptane (17/83) | Supernatant from Sample 22.2; | Clear | — |
| | crystallization in freezer | | |
| IPA/heptane (67/33) | Supernatant from Sample 22.1; | Clear | — |
| | crystallization in freezer | | |
| MeOH | Slow evaporation | Glass | — |
| | (concentration 19 mg/mL) | | (Sample 19.1) |
| | Slow evaporation | Glass | — |
| | (concentration 26 mg/mL) | | (Sample 19.2) |
| MeOH/chloroform | Slow evaporation | Film, no BE | — |
| (17/83) | | | (Sample 19.3) |
| 2-MeTHF | Slow evaporation | Film, no BE | — |
| | | | (Sample 19.4) |
| | Slow evaporation (3 mg scale) | BE | Fumarate Form C, selected |
| | | | peaks slightly shifted + Kapton |
| | | | peak at 5, 15, and 29° 2θ |
| MIBK | Crystallization in freezer (4 mg | Clear | — |
| | scale) | | |
| MTBE | Supernatant from Sample 22.3; | Clear | — |
| | crystallization in freezer | | |
| TFE | Fast evaporation | Film, no BE | — |
| | | | (Sample 19.5) |
| — | Water vapor stress, 1 day | — | Fumarate Form C, selected |
| | | | peaks, slightly shifted |
| — | 75% RH stress, 2 days | — | Fumarate Form C |

BE = birefringence/extinction;
SCXRD = single crystal X-ray diffraction

Crystallization of glasses and films obtained from the kinetic experiments was conducted via stirring for approximately 22 days (Table 23). Sample numbers reference Table 22.

TABLE 23

| Starting | Conditions | | |
|---|---|---|---|
| Material | (Solvent Ratio v %) | Observations | XRPD Results |
| Sample 19.1 | MTBE slurry, RT, 22 days | Dry, yellow solids | Fumarate Form H + |
| | | | Kapton peak at 5° 2θ |
| Sample 19.2 | MIBK slurry, RT, 22 days | Yellow solids | Fumarate Form D + |
| | | | Kapton peak at 5° 2θ |
| Sample 19.3 | Water slurry, RT, 22 days | Yellow solids | Fumarate Form J, |
| | | | disordered + Kapton |
| | | | peak at 5° 2θ |
| Sample 19.5 | IPA/heptane (22/78) slurry, | Yellow solids | Fumarate Form E + |
| | RT, 22 days | | Kapton peak at 5° 2θ |
| Sample 19.4 | 2-BuOH slurry, RT, 22 days. | No solids | — |
| | Concentrated by slow | | |
| | evaporation. | | |

Table 24 provides a summary of the characterization data for the materials produced from this experiment.

TABLE 24

| Material Designation | Analytical Technique | Results |
|---|---|---|
| Fumarate Form E | XRPD | Crystalline, single crystalline phase, unit cell volume |
| (produced in IPA) | | from XRPD indexing is consistent with unsolvated |
| | | mono-fumarate salt. Broad feature near 5° 2θ due to |
| | | Kapton film. |
| | PLM | Solids were placed on a microscope slide, covered by |
| | | a coverslip. Mineral oil was added through capillary |
| | | action. |
| | | Loose and agglomerated particles with BE. Individual |
| | | particles appear primarily as blades with some |
| | | particles being flaky and anhedral. Various sizes of |
| | | agglomerates, which appear to be hard upon light |
| | | pressure. |
| | ¹H NMR (6 days after XRPD) | Consistent with Compound 1 fumarate with 1:1 |
| | | stoichiometry. Organic solvents are not apparent. |
| | TGA (10 days after XRPD) | Weight loss, onset at 80° C. |
| | DSC (10 days after XRPD) | Strong endotherm, onset at 147° C. and peak max at |
| | | 156° C. |
| Fumarate Form E | XRPD | Crystalline, minor shifts of selected XRPD peaks |
| (produced in 2-BuOH) | | compared to Fumarate Form E generated in IPA. |
| | | Broad feature near 5° 2θ due to Kapton film. |
| | ¹H NMR (1 month after XRPD) | Consistent with Compound 1 fumarate with 1:1 |
| | | stoichiometry. Trace 2-BuOH possible. |
| | TGA (3 weeks after XRPD) | 6.3 wt % loss, 29-92° C. (equivalent to 2 mol water). |
| | | Weight loss, onset at 179° C. |
| | DSC (3 weeks after XRPD) | Weak broad and asymmetric endotherm, peak max at |
| | | 70° C. Strong endotherm, onset at 147° C. and peak |
| | | max at 155° C. |
| Fumarate Form E | XRPD | Crystalline, XRPD peak shifts are not apparent |
| (produced in | | compared to Fumarate Form E generated in IPA. |
| NMP/water 40/60) | | Broad feature near 5° 2θ due to Kapton film. |
| | ¹H NMR (1 month after XRPD) | Consistent with Compound 1 fumarate with 1:1 |
| | | stoichiometry. NMP (1 mol/mol), based on peaks at |
| | | 3.3, 2.2, and 1.9 ppm. |
| | TGA (3 weeks after XRPD) | 1.5 wt % loss, 35-91° C. (equivalent to 0.5 mol water) |
| | | Weight loss, onset at 178° C. |
| | DSC (3 weeks after XRPD) | Broad endotherm, peak max at 80° C. |
| Fumarate Form D | XRPD | Crystalline, single crystalline phase, unit cell volume |
| | | from XRPD indexing is consistent with mono-MIBK |
| | | solvate of mono-fumarate salt of Compound 1. Broad |
| | | feature near 5° 2θ due to Kapton film. |
| Fumarate Form D | XRPD | Crystalline, single crystalline phase |
| | ¹H NMR (6 days after XRPD) | Consistent with Compound 1 fumarate with 1:1 |
| | | stoichiometry. MIBK (0.7 mol/mol), based on peaks at |
| | | 2.3, 2.1, 2.0, and 0.9 ppm. |
| | TGA (10 days after XRPD) | 1.0 wt % loss, 48-155° C. (equivalent to 0.06 mol of |
| | | MIBK or 0.5 mol of water). |
| | | Weight loss, onset at 180° C. |
| | DSC (10 days after XRPD) | Strong endotherm, onset at 135° C. and peak max at |
| | | 146° C. |
| Fumarate Form F | XRPD | Crystalline, single crystalline phase, unit cell volume |
| | | from XRPD indexing is consistent with mono- |
| | | tBuOAc solvate of mono-fumarate salt of Compound |
| | | 1. Broad feature near 5° 2θ due to Kapton film. |
| | ¹H NMR (6 days after XRPD) | Consistent with Compound 1 fumarate with 1:1 |
| | | stoichiometry. tBuOAc (0.7 mol/mol), based on peaks |
| | | at 1.9 and 0.39 ppm. |
| | TGA (10 days after XRPD) | 11.7 wt % loss, 48-157° C. (equivalent to 0.6 mol |
| | | tBuOAc) |
| | | Weight loss, onset at 180° C. |
| | DSC (10 days after XRPD) | Small broad endotherms, peak max at 97° C. |
| | | Strong endotherm, onset at 137° C. and peak max at |
| | | 145° C. |
| Fumarate Form G | XRPD | Crystalline, single crystalline phase, unit cell volume |
| | | from XRPD indexing is consistent with mono-ACN |
| | | solvate of mono-fumarate salt of Compound 1. |
| | ¹H NMR (6 days after XRPD) | Consistent with Compound 1 fumarate with 1:1 |
| | | stoichiometry. ACN (0.3 mol/mol), based on peak at |
| | | 2.07 ppm. |
| | TGA (10 days after XRPD) | 1.3 wt % loss, 48-149° C. (equivalent to 0.2 mol ACN). |
| | | Weight loss, onset at 180° C. |
| | DSC (10 days after XRPD) | Strong endotherm, onset at 140° C. and peak max at |
| | | 149° C. |
| Fumarate Form C | XRPD | Crystalline, single crystalline phase, unit cell volume |
| | | from XRPD indexing is consistent with mono- |
| | | fumarate salt of Compound 1 containing up to 2 mol |
| | | of water. |

Salt formation experiments were also performed by stirring mixtures of Amorphous Compound 1 and fumaric acid (1:1.2 ratio). Solids precipitated were isolated via centrifugation with filtration. Results are summarized in Table 25.

TABLE 25

| Conditions (Solvent Ratio v %) | XRPD Results |
|---|---|
| 1) Suspended 81 mg of Compound 1 and 24 mg of fumaric acid in 2 | Fumarate Form E, |
| mL of IPA with stirring (clear) | slightly disordered + |
| 2) Added 1 mL of heptane (clear) | Kapton peak at 5° 2θ |
| 3) Placed in freezer, 1 day | (Sample 22.1) |
| 4) Isolated cold via centrifugation with filtration (greasy pasty solids) | |
| 5) Air dried (loose solids) | |
| 1) Suspended 81 mg of Compound 1 and 26 mg of fumaric acid in 1 | Fumarate Form C, |
| mL IPA with stirring and placed on hot plate (clear) | shifted |
| 2) Added 5 mL of heptane by aliquots (some precipitation) | (Sample 22.2) |
| 3) Cooling to RT (partially oiled out) | |
| 4) Sonicated, 1 hour (solids, no oil) | |
| 5) Stirring, RT, 1 day | |
| 6) Isolated via centrifugation with filtration (greasy, pasty solids) | |
| 7) Air dried (loose solids) | |
| 1) Suspended 80 mg Compound 1 and 26 mg fumaric acid in 2 mL of | Fumarate Form H + |
| MTBE with stirring (some dissolution, then additional precipitation) | fumaric acid |
| 2) Stirring, RT, 1 day | (Sample 22.3) |
| 3) Isolated via centrifugation with filtration (loose solids) | |

1. A crystalline solid form of Compound 1 Fumarate: 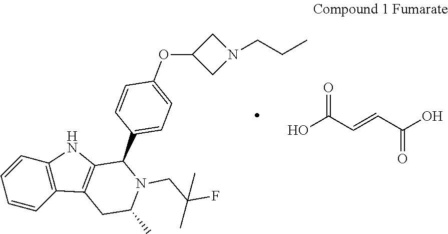 [SMILES: CCCN1CC(Oc2ccc([C@@H]3c4[nH]c5ccccc5c4C[C@@H](C)N3CC(C)(C)F)cc2)C1 ; O=C(O)/C=C/C(=O)O]
wherein the solid form is Form E.

2. The crystalline solid form of claim 1, wherein the crystalline solid form is characterized by one or more peaks in its XRPD pattern selected from those at about 5.83, about 7.03, about 8.69, about 12.88, about 13.43, about 14.68, about 15.65, about 16.65, and about 18.46 degrees 2-theta.

3. The crystalline solid form of claim 1, wherein the crystalline solid form is characterized by peaks in its XRPD pattern at about 5.83, about 7.03, about 8.69, about 12.88, about 13.43, about 14.68, about 15.65, about 16.65, and about 18.46 degrees 2-theta.

4. The crystalline solid form of claim 1, wherein the crystalline solid form is characterized by peaks in its XRPD pattern at substantially all of:
Position ± 0.2 (degrees 2-Theta)
5.83
7.03
8.69
10.91
12.88
13.43
14.68
15.65
16.08
16.65
17.72
18.46
18.88
19.63
19.99
21.73
22.00
22.31
23.83
24.63
25.02
26.48
27.37
28.60
29.32
29.81
30.05
35.69
39.70
40.32
40.97

5. The crystalline solid form of claim 1, wherein the crystalline solid form is characterized by one or more of the following:
(i) an XRPD pattern substantially similar to that depicted in FIG. 1 and/or FIG. 3;
(ii) a TGA pattern substantially similar to that depicted in FIG. 2 and/or FIG. 4; and
(iii) a DSC pattern substantially similar to that depicted in FIG. 2 and/or FIG. 5.

6. A pharmaceutical composition comprising the crystalline solid form of any one of claim 1-5 and a pharmaceutically acceptable carrier.

7. The pharmaceutical composition of claim 6, wherein the pharmaceutical composition is a solid.

8. The pharmaceutical composition of claim 6 or 7, wherein the pharmaceutical composition is formulated for oral administration.

9. A method of inhibiting an estrogen receptor or mutant thereof in a biological sample comprising contacting the biological sample with the crystalline solid form of any one of claims 1-5.

10. A method of inhibiting an estrogen receptor or mutant thereof in a patient comprising contacting the patient with the solid form of any one of claims 1-5.

11. A method of treating a disease, disorder, or condition associated with the estrogen receptor in a patient, comprising administering to the patient a therapeutically effective amount of the crystalline solid form of any one of claims 1-5.

12. The method of claim 11, wherein the disease, disorder, or condition is selected from the group consisting of breast cancer, bone cancer, lung cancer, colorectal cancer, endometrial cancer, prostate cancer, ovarian cancer, vaginal cancer, endometriosis, and uterine cancer.

13. The method of claim 12, wherein the disease, disorder, or condition is breast cancer.

14. The method of any one of claims 11-13, further comprising administering another anti-cancer agent.

15. The method of claim 14, wherein the anti-cancer agent is a CDK4/6 inhibitor, a PI3KCA inhibitor, or an mTOR inhibitor.

16. A method of preparing a crystalline solid form of Compound 1 Fumarate: 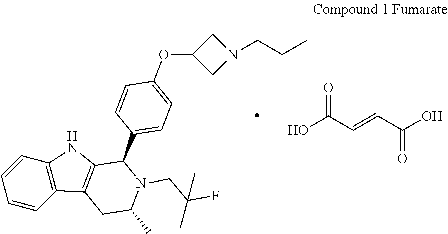 [SMILES: CCCN1CC(Oc2ccc([C@@H]3c4[nH]c5ccccc5c4C[C@@H](C)N3CC(C)(C)F)cc2)C1 ; O=C(O)/C=C/C(=O)O]
wherein the crystalline solid form is Form E,
the method comprising contacting Compound 1: 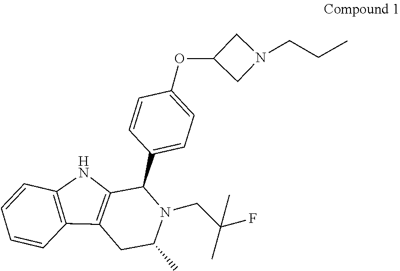 [SMILES: CCCN1CC(Oc2ccc([C@@H]3c4[nH]c5ccccc5c4C[C@@H](C)N3CC(C)(C)F)cc2)C1]
with fumaric acid in a suitable solvent to provide Compound 1 Fumarate Form E.

17. The method of claim 16, wherein the suitable solvent is isopropanol.

18. The method of claim 16 or 17, comprising dissolving Compound 1 in the suitable solvent and adding the fumaric acid in portions.

19. The method of any one of claims 16-18, comprising adding seed crystals of Compound 1 Fumarate Form E.

20. The method of claim 18 or 19, wherein at least one portion of fumaric acid is added before addition of the seed crystals.

21. The method of any one of claims 18-20, wherein at least one portion of fumaric acid is added after addition of the seed crystals.

22. A composition comprising a crystalline solid form of Compound 1 Fumarate: 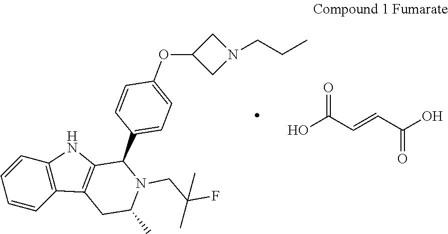 [SMILES: CCCN1CC(Oc2ccc([C@@H]3c4[nH]c5ccccc5c4C[C@@H](C)N3CC(C)(C)F)cc2)C1 ; O=C(O)/C=C/C(=O)O]
wherein the solid form is Form E, and
wherein the composition is substantially free of impurities.

23. A composition comprising a crystalline solid form of Compound 1 Fumarate: 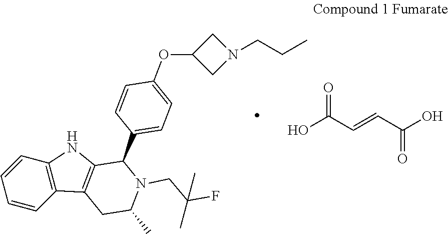 [SMILES: CCCN1CC(Oc2ccc([C@@H]3c4[nH]c5ccccc5c4C[C@@H](C)N3CC(C)(C)F)cc2)C1 ; O=C(O)/C=C/C(=O)O]
wherein the solid form is Form E, and
wherein the composition is substantially pure.

24. A composition comprising a crystalline solid form of Compound 1 Fumarate: 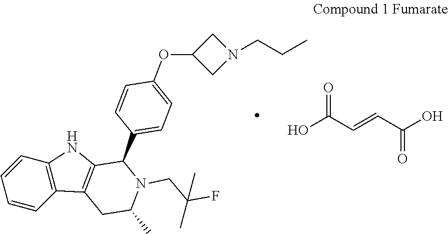 [SMILES: CCCN1CC(Oc2ccc([C@@H]3c4[nH]c5ccccc5c4C[C@@H](C)N3CC(C)(C)F)cc2)C1 ; O=C(O)/C=C/C(=O)O]
wherein the solid form is Form E, and
wherein the composition is substantially free of an amorphous solid form.
